(19) United States
(12) Patent Application Publication
(10) Pub. No.: US 2011/0230433 A1
(43) Pub. Date: Sep. 22, 2011

(54) COMPOSITIONS AND METHODS FOR TREATMENT OF CANCER

(75) Inventors: Lawrence A. Loeb, Bellevue, WA (US); Edward J. Fox, Seattle, WA (US); Marc J. Prindle, Seattle, WA (US)

(73) Assignee: UNIVERSITY OF WASHINGTON, Seattle, WA (US)

(21) Appl. No.: 13/049,390

(22) Filed: Mar. 16, 2011

Related U.S. Application Data
(60) Provisional application No. 61/314,477, filed on Mar. 16, 2010.

(51) Int. Cl. A61K 31/708; A61K 31/7072; C12N 15/01; A61P 35/00

(52) U.S. Cl. 514/45; 514/50; 435/441

(57) ABSTRACT

Described herein are novel methods and compositions for treatment of cancer by increasing the mutation rate of cancer cells beyond an error threshold, over which the cancer cells are no longer viable. In particular, mutagenic compounds such as nucleoside analogs for treatment of cancer are also described herein.

### CROSS-REFERENCE TO RELATED APPLICATIONS

This application claims benefit under 35 U.S.C. §119(e) of Provisional Application No. 61/314,477, filed on Mar. 16, 2010, the contents of which is incorporated herein by reference in its entirety.

### GOVERNMENT SUPPORT

This invention was made with government support under Grant No. 5RO1CA102029 and 5RO1CA115802 awarded by the National Institutes of Health (NIH). The government has certain rights in the invention.

### FIELD OF THE INVENTION

The present invention generally relates to cancer therapies. Described herein are compositions and methods for treatment of cancer. In particular, compositions and methods for targeting the mutator phenotype in cancer cells are described herein

### BACKGROUND OF THE INVENTION

Cancer is characterized by mutations that enable cells to divide where and when they should not, invasion, and metastasis. It is believed that the tens of thousands of genetic alterations in most human cancers do not result from the low inherent mutation rates exhibited by normal human cells; instead, cancer cells express a mutator phenotype [1]. The underlying premise is that mutation rates in normal human cells are insufficient to account for the multiple mutations found in human cancers, and that cancers must therefore exhibit an increased mutation rate early in their evolution. This concept was initially formulated based on identification of mutations in DNA polymerases that render them error-prone, and mutations in enzymes involved in DNA repair that decrease the ability of cells to remove potentially mutagenic DNA lesions [2]. As a result, there is believed to be increased genomic instability with the acquisition of mutations in oncogenes and tumor suppressor genes. With the increased number of genes shown to be required for guaranteeing genetic fidelity, the mutator phenotype hypothesis has been broadened to include any gene involved in the maintenance of genetic stability [3].

Evidence for the involvement of large numbers of mutations in tumor progression has been based mainly on chromosomal aberrations and molecular features of certain hereditary cancers. Early indications of a central role of genome alterations in cancer development emerged in the late nineteenth and early twentieth centuries from studies by von Hansemann and Boveri. Techniques such as array comparative genomic hybridization and spectral karyotyping have more recently enabled higher resolution than early cytological observations, and have been used to demonstrate that individual metastatic cancer cells harbor a diverse spectrum of unique chromosomal aberrations. Using complementary techniques, Stoler et al. estimated that the mean number of genomic events per carcinoma cell is greater than 10,000. Additional evidence for thousands of mutations in cancer cells came from the observation of changes in the length of microsatellites in tumor DNA from patients with Lynch syndrome (also known as hereditary nonpolyposis colorectal cancer or HNPCC). These patients harbor mutations in mismatch repair (MMR) genes, and as a result accumulate thousands of point mutations as well as mutations in as many as 100,000 repetitive sequences per cancer genome. Microsatellite instability has also been detected in sporadic tumors without mutations in MMR genes, and in premalignant conditions associated with chronic inflammation. These findings indicate that changes in cellular environments, such as hypoxia, can result in a transient deficiency in MMR and give rise to a mutator phenotype. Alterations in the length of poly(dG) repeats in otherwise normal appearing colonic epithelium have even been shown to identify colon cancers at distant sites.

The importance of maintaining genome integrity in preventing tumorigenesis is highlighted by a number of inherited diseases which are associated with elevated risks of specific cancers, and are caused by germline mutations in genes involved in DNA repair. This association between DNA repair and suppression of carcinogenesis was established, for example, by the seminal findings of the UV-induced DNA damage repair defects in patients with xeroderma pigmentosum. Inherited defects in components of several other DNA-repair pathways also underlie a variety of cancer predisposing syndromes including: mismatch repair (Lynch syndrome), base excision repair (MYH-associated polyposis), homologous recombination (early onset breast cancer), non-homologous DNA end joining (Lig4 syndrome), and translesion synthesis (xeroderma pigmentosum variant). Hereditary mutations in other genes that are believed to be required for DNA maintenance are also associated with cancer. For instance, mutations in TP53 are found in Li-Fraumeni syndrome, a highly cancer-prone condition most frequently associated with sarcomas and breast adenocarcinomas. Additionally, polymorphisms in a large number of genetic stability genes, including OGG1 and XRCC1, are emerging as risk alleles for many cancers.

Recent evidence strongly supporting the mutator phenotype hypothesis comes primarily from three sources: first, mathematical models that quantitatively predict the efficiency of carcinogenesis with and without a mutator phenotype, indicate that mutator mutations are required for the multiple steps involved in tumor progression; second, human tumors have been shown to have a high frequency of random single base substitutions; and third, DNA sequencing projects have now catalogued large numbers of clonal mutations in individual tumors. These sequencing studies, in particular, have shown that the mutational load in cancer is substantial and highly heterogeneous.

The mathematical models of particular importance are the ones that address the likelihood that a tumor would evolve by a mutator pathway. If only one to three driving or initial mutations are required, increased mutation rates in cancer cells are not required to account for the emergence of a tumor [4]. However, if a large number of driving mutations are required, mutator mutations will greatly accelerate carcinogenesis, particularly if they are early events. These models also consider the impact of increased cell death [5], and of clonal expansion [6].

Methods have been recently developed to measure the number of random mutations in a tumor. The International Cancer Genome Consortium, formed in 2008, is currently coordinating efforts to sequence 500 tumors from each of 50 cancer types. It includes two older large-scale projects: the Cancer Genome Atlas and the Cancer Genome Project. Both of these projects were initially undertaken with the expectation that exhaustive sequencing of tumor cell DNA would reveal a small number of key mutations in each cancer type, which would then serve as targets for novel, molecularly directed anticancer therapies. The opposite, however, has been found: very few genes are commonly mutated in human cancers. While early cancer genome studies focused primarily on protein coding regions of the genome, the most recent phase of these studies has seen the whole genome characterization of a number of specimens, including acute myeloid leukemia, small cell lung cancer, melanoma, breast cancer, and non-small cell lung cancer. These later studies have unequivocally established that tens of thousands of clonal mutations are present in each cancer genome. As predicted by the mutator phenotype hypothesis, mutations were found to be distributed throughout the nuclear genome of these tumors, with on average one to ten mutations per million base pairs.

Most of the mutations identified by these studies do not appear to be causally involved in the pathogenesis of cancer and only a small subset of the nonsynonymous substitutions are even believed to have been affected by selection. Such non-causal mutations are termed “passenger” mutations. While the substitution trends can partially reflect underlying mutational processes, their distribution can also correspond to hotspots for mutagenesis, such as in regions of DNA that can assume secondary structures, e.g., hairpins, triplestranded or quadruplex DNA. Another prediction of the mutator phenotype hypothesis is that subclonal mutations are present in large numbers. Many of these mutations are likely to be in the “driver” genes identified by current methods of DNA sequencing. In addition to the extensive clonal heterogeneity being uncovered, additional mutational diversity has been found to exist within individual tumors themselves. Thus, a large number of subclonal and random mutations are also present, conferring a unique mutational signature on each cancer cell. This mutational complexity within cancer cells provides a genetic basis for the wide variations observed in tumor behavior and responsiveness to therapy, between and within individuals.

Some nucleoside analogs have been used for the treatment of cancer. One of the most successful agents for a variety of cancers, such as colorectal cancer, is the pyrimidine analog 5-fluorouracil, which selectively kills cancer cells by inhibiting ribonucleotide reductase, leading to replicative stress, changes in nucleoside pools in cells, DNA damage, and cell death. Many other common agents used in cancer chemotherapy also act at the level of DNA metabolism, creating blocking lesions in DNA that stall DNA polymerases, or directly poisoning topoisomerases, leading to double strand breaks. However, these chemotherapeutic agents, including the currently available nucleotide analogs, can increase some cancer cells' drug resistance and thus mitigate the initial effectiveness of the drugs.

### SUMMARY OF THE INVENTION

Provided herein are compounds, compositions and methods for treatment of cancer. The increase in the mutation rate of cancer cells beyond a critical threshold level can result in reduced viability of cancer cells. Accordingly, in one aspect, described herein are mutagenic compounds for increasing the mutation frequency of a mammalian cell, e.g., a mammalian cancer cell. The mutagenic compounds described herein, when in contact with a mammalian cell, increase the mammalian cell's mutation frequency by at least about two-fold, relative to the mutation frequency of the mammalian cell when not in contact with the mutagenic compounds.

Another aspect described herein includes methods of increasing mutation frequency in a genome of a cancer cell. The methods comprise contacting a cancer cell with one or more mutagenic compounds, which increase the mutation frequency of a cancer cell or progeny cell thereof by at least about two-fold relative to the mutation frequency of the cancer cell or progeny cell thereof when not contacted by the one or more mutagenic compounds.

In some embodiments, the increase in mutation frequency of the cancer results in lethal mutagenesis of the cancer cell or progeny cell thereof.

While cancer cells generally have an inherently high mutation rate, increasing that mutation rate in the genome of the cancer cells can be used to treat the tumor. An increase in the already-high mutation rate in the genome of cancer cells can be used to induce lethal mutagenesis in the cancer cells, such that the cancer cells are killed and the tumor is abolished. It is contemplated that any compound that increases the frequency of mutations in a cell can be used in the therapeutic methods described herein. For example, compounds that increase the frequency of mutations by inhibition of DNA repair mechanisms, by reducing the fidelity of DNA replication, by influencing the balance of the nucleotide pool and/or by becoming incorporated into the genome, are specifically contemplated. In some embodiments, the mutagenic compounds increase the mutation frequency of the cancer cell by increasing non-complementary nucleoside mispairings in the cancer cell. In some embodiments, at least one mutagenic compound is incorporated into the genome of the cancer cell or progeny cell thereof by a replicative DNA polymerase in the cancer cell. In other embodiments, a mutagenic compound as described herein does not inhibit replication or extension of a nucleic acid by a DNA polymerase. In some embodiments, a mutagenic compound for lethal mutagenesis can be identified by: (a) assessing the ability of candidate mutagenic compounds to become incorporated into DNA and induce mutagenesis and cell death in cultured cells; and (b) introducing chemical modifications at various sites on the subject mutagenic compounds to enhance its stability, its incorporation rate, and its ability to introduce mutations in a given cell.

Methods of treating cancer in a subject in need thereof are also described herein. The methods include administering to a subject having a cancer or a tumor a pharmaceutical composition comprising at least one mutagenic compound as described herein. In some embodiments, the methods further comprise administering to the subject an anti-cancer agent. In such embodiments, the anti-cancer agent to be administered is different from the mutagenic compound first administered to the subject. The anti-cancer agent can be another mutagenic compound different from the first mutagenic compound administered to the subject, or other therapeutics, such as non-mutagenic anti-cancer agents. In some embodiments, the subject having a cancer or a tumor has been diagnosed as therapy-resistant, e.g., resistant to chemotherapy. In contrast to conventional treatments that include administration of analogs that are either inhibitors of ribonucleotide reductase, or that are incorporated as blocking lesions in DNA, the methods presented herein provide a high frequency of non-complementary base-pairings by the administered mutagenic compounds (e.g., mutagenic nucleoside analog) which can increase the mutation rate of the cancer cells. Given the high number of existing mutations in cancer cells, increasing the mutation rate can increase the likelihood of achieving a lethal or synthetic lethal mutation in a given cell.

A mutagenic compound can comprise a nucleoside analog or a nucleoside mimic. In various embodiments, the mutagenic compounds described herein comprise a nucleoside analog. Exemplary nucleoside analogs include, but are not limited to, O⁴-methyl thymidine, O⁴-ethyl thymidine, 8-hydroxy deoxyguanosine, Etheno-dG, Etheno-dA, or Etheno-dC, O⁶-methyl dG, O⁶-ethyl dG, 5-OH-methyl cytidine, 2-amino-9-(β-D-2′-deoxyribfuranosyl)purine, 3-(2′-Deoxy-β-D-2-ribofuranosyl)pyrido[2,3-d]pyrimidine-2,7(8H)-dione (dF), and 2′-Deoxyinosine.

While the methods described herein include administration of mutagenic nucleosides or deoxynucleosides, it is understood that these methods can also include, in addition to or as an alternative, the administration of mutagenic nucleotides or mutagenic deoxynucleosides.

### BRIEF DESCRIPTION OF THE DRAWINGS

FIG. 1 is a graphical representation of the outcome of targeting the mutator phenotype in cancer in accordance with one or more embodiments described herein. Over time, untreated cancer accumulates mutations, contributing to heterogeneity and promoting carcinogenesis. A cancer initiating event occurs at 0 years. Diversity of the cancer cell population is represented by different shapes of nuclei in the cells, and tumor burden is represented by the size of the cell clusters. Reducing the rate of mutation, or Treatment by Delay, can prolong the time to tumorigenesis by a significant degree in some cancers. Accelerating the accumulation of mutations beyond the rate favored by selection, or Lethal Mutagenesis, can rapidly drive cancer cells over an error threshold, increasing the chance that most cancer cells will acquire a lethal mutation.

FIGS. 2A to 2D show examples of nucleoside analogs that can be used for the purposes described herein. Shown is a library of modified 2′ deoxyguanosine analogs involving (FIG. 2B) O⁶-, (FIG. 2C) N³- and (FIG. 2D) C⁸-atoms of guanine (FIG. 2A).

FIG. 3 is a schematic showing original and simplified Random Mutation Capture (RMC) assays. To ensure complete digestion of non-mutant (wild type) sequences, the original assay employed a series of target enrichment steps. By limiting the amount of DNA to be digested by the restriction enzyme reaction, these enrichment steps can be omitted. This has the added advantage of allowing multiple target sequences to be interrogated simultaneously and reduces the overall amount of DNA required to perform the assay.

### DETAILED DESCRIPTION OF THE INVENTION

The effectiveness of conventional chemotherapeutics for treatment of cancer can decrease over time when cancer cells develop drug resistance to chemotherapy, e.g., by increasing mutation rate on their own (but below an error threshold) and thus becoming more malignant. As such, there is an urgent need to develop a more effective therapeutic for treatment of cancer.

While the extensive genetic variation within a cancer cell population represents clinically important consequences of the mutator phenotype, the mutation burden of cancer as a therapeutic target has never been explored. The mutation burden of cancer cells can itself present a therapeutic target strategy. Without wishing to be bound or limited by theory, genetic instability in cancer cells likely exists at a threshold, such that appropriate levels of instability allow selection barriers to be overcome, but excessive instability can lead to extinction of the unstable clone. Modulating the mutation frequency of a cancer genome, e.g., by increasing the mutation burden beyond an error threshold for tumor cell viability, can be employed to decrease the overall fitness of the tumor cell population and eventually abolish the tumor, as described herein.

Accordingly, some aspects described herein are generally related to methods and compositions that increase mutation frequency in a genome of a cancer cell or progeny thereof. Other aspects described herein are generally directed to methods and compositions for treating cancer in a subject. In one aspect, the methods and compositions described herein are directed to the use of a mutagenic compound (e.g., mutagenic nucleoside analog) as a therapeutic agent for treatment of cancer. Specifically, the mutagenic compound (e.g., mutagenic nucleoside analog) administered for treatment of cancer can increase the mutation frequency of a cancer cell by at least about 2-fold. In some embodiments, the administration of such mutagenic compound provides non-complementary base-pairings for increasing the mutation rate in a cancer cell. In some embodiments, the mutagenic compound is incorporated and extended by DNA polymerase in a cancer cell. In alternative embodiments, the mutagenic compound is not incorporated by DNA polymerase in a cancer cell. In some embodiments, the mutagenic compound is a nucleoside analog. Examples of such nucleoside analogs that can be used for the compositions and methods described herein include, but are not limited to, O⁴-methyl thymidine, O⁴-ethyl thymidine, 8-hydroxy deoxyguanosine, Etheno-dG, Etheno-dA, Etheno-dC, O⁶-methyl dG, O⁶-ethyl dG, 5-OH methyl cytidine, 2-amino-9-(β-D-2′ deoxyribfuranosyl)purine, 3-(2′-Deoxy-β-D-2-ribofuranosyl)pyrido[2,3-d]pyrimidine-2,7(8H)-dione (dF) and 2′-deoxyinosine.

### Expression of a Mutator Phenotype

In contrast to the rarity of mutations in normal cells, the genomes of cancer cells contain multiple changes. Theodor Boveri first suggested that malignancy resulted from a disturbance of chromosomal balance and, although specific chromosomal alterations are diagnostic of only a few types of cancer, i.e. their presence is indicative of having a certain cancer type, chromosomal aberrations are observed in most tumors [7]. Multiple chromosomal alterations occur in both benign and malignant tumors and include deletions, additions, amplifications, and translocations, frequently involving millions of nucleotides [8]. Studies on DNA copy number and loss of heterozygosity (LOH) have established that individual tumors from a variety of cancer types can contain more than 30 independent clonal chromosomal alterations [9, 10]. Primary adenocarcinomas of the breast and colon, for example, frequently harbor more than 20 different chromosomal alterations [11, 12], and individual disseminated breast cancer cells have been shown to carry a diverse spectrum of chromosomal abnormalities distinct from that of the primary cancer [13]. These results indicate that chromosomal abnormalities continually accumulate during tumorigenesis even subsequent to the clinical appearance of a tumor or cancer cells.

Definitive evidence for multiple nucleotide sequence changes within the genomes of cancer cells came initially from findings in Lynch syndrome (also known as hereditary non-polyposis colorectal cancer (HNPCC) syndrome) and in a subset of sporadic colorectal tumors [14-16]. In hereditary colorectal cancers, which involve mutations in mismatch repair genes, microsatellites and other repetitive sequences throughout the genome display extensive somatic length variations. Based on the frequency of mutations in a limited number of microsatellites examined, it is estimated that thousands of loci are mutated within the DNA of each tumor cell [17]. Repetitive DNA sequences are mutational “hotspots” in which slippage by DNA polymerases exceeds the capacity for correction by mismatch repair mechanisms. This results in elongation or shortening of the repeats, and can cause frame-shift mutations within coding sequences or alter the spacing between DNA regulatory elements [18]. In addition, this mismatch repair mutator phenotype results in a dramatic increase in the frequency of random somatic point mutations [19]. In chronic ulcerative colitis (UC), the presence of microsatellite mutations at sites that are distant from areas of dysplasia provides a powerful marker to foretell the likelihood for the development of malignancy, and offers the potential to map cell lineages [20]. While high levels of microsatellite instability also occur in 10-15% of sporadic colorectal cancer, length alterations of repetitive sequences are likely present in all tumors, provided enough markers are assayed [21].

The recent effort to characterize the human cancer genome has yielded a wealth of information about clonal mutations present in a variety of human tumors. Complete sequencing of all predicted human coding exons in breast and colon cancer samples identified medians of 84 and 76 non-silent mutations per tumor, respectively [12, 22]. While almost 10% of the 18,197 genes studied were detectably mutated in at least one specimen, each tumor displayed a unique and highly varied profile of mutated genes. Contrary to expectations, other than the few genes previously known to be frequently mutated in these cancer types (e.g., TP53 and APC), no new mutated genes were identified that occurred at a high prevalence. It was thus determined that genomes of cancer cells comprise a larger number of infrequently mutated genes, each providing a small fitness advantage, and few commonly mutated genes [12]. Consequently, the wide variation in tumor behaviors and responsiveness to therapy can be due to clonal mutational heterogeneity in cancer cells. For example, while chemotherapy can be effective to treat some tumor cells within a tumor, a small population of tumor cells can also be selected for malignancy, e.g., with an increased drug resistance to chemotherapy.

Follow-up studies on diverse cancer types have generated extensive catalogs of clonal somatic point mutations in a variety of tumor types (Table 1). These studies indicate that all cancer types studied to date display significant heterogeneity at the genetic level, in part as a consequence of their underlying mutator phenotypes [23]. Individual solid organ tumors typically harbor non-silent clonal genetic alterations in more than 50 different genes. Despite the fact that a small number of these genes are mutated in a high proportion of cancers, the prevalence at which the majority are mutated within different tumors of the same cancer type is low. These results are not in accord with the classical paradigm, in which a small number of key genes, mutated in a linear temporal sequence, are believed to drive tumorigenesis.

TABLE 1
Summary of data from selected cancer genome sequencing studies.

| | | | Tumor | Non-Silent | |
|---|---|---|---|---|---|
| | | Genes | Muations | Mutations | |
| Tumor | (n) | Sequenced | (Tumor) | per Tumor | References |
| Breast | 11 | 18,191 | 1,243 | 84 | [12, 22] |
| Colorectal | 11 | | 942 | 76 | |
| Diverse | 210 | 518 | 798 | NA | [60] |
| Pancreatic | 24 | 20,661 | 1,163 | 48 | [10] |
| Glioblastoma | 210 | 20,661 | 748 | 47 | [61] |
| Glioblastoma | 91 | 601 | 453 | NA | [62] |
| Lung | 188 | 623 | 1,013 | NA | [63] |
| Acute | 1 | Whole | 500-1,000 | 10 | [24] |
| Myeloid | | Genome | | | |
| Leukemia | | | | | |
| Acute | 1 | Whole | 750 | 12 | [25] |
| Myeloid | | Genome | | | |
| Leukemia | | | | | |
| Lung | 1 | Whole | >33,345 | 188 | [27] |
| | | Genome | | | |
| Malignant | 1 | Whole | >22,910 | 100 | [26] |
| Melanoma | | Genome | | | |
| Small Cell | 1 | Whole | 22,910 | 101 | [71] |
| Lung Cencer | | Genome | | | |
| Melanoma | 1 | Whole | 33,345 | 182 | [72] |
| | | Genome | | | |
| Breast | 3 | Whole | 27,173 | ~200 | [73] |
| Cancer | | Genome | | | |
| Non-Small | 1 | Whole | 50,675 | 302 | [74] |
| Cell Lung | | Genome | | | |
| Cancer | | | | | |
| Normal | | Whole | 70 | <<1 | [75] |
| human | | Genome | | | |
| (between | | | | | |
| generations) | | | | | |

These studies focused exclusively on identifying mutations in exons of known protein-coding genes. With increasing recognition of the importance of non-coding DNA, including regulatory regions and sequences that encode noncoding RNAs, it is believed that the total number of functionally consequential clonal mutations is greater than those identified in coding sequences of genes. The first unbiased whole genome sequencing study was performed on an acute myeloid leukemia (AML) genome and was the first complete description of all clonal sequence alterations within a single cancer [24]. By exhaustively interrogating the cancer genome and, importantly, the paired normal genome, i.e., genome of non-cancerous/non-tumor cells, of a single AML type M1 patient using massively-parallel sequencing, 500 to 1,000 somatic mutations were identified, of which less than 2 percent were in protein-coding genes. Although the overall number of protein-coding mutations (10) is fewer than that described for solid organ tumors (Table 1), similar to the pattern observed in other cancer types, none of the 8 newly identified genes were found to be mutated in a further 187 cases of AML. These results have been replicated in a follow-up study confirming that, genome-wide, approximately 750 clonal mutations are present in the minimally cytogenetically altered AML-M1 cancer genome [25]. A similar pattern has also recently emerged for the genomes of solid tumors; unbiased whole genome sequencing has uncovered greater than 20,000 mutations in the cancer genomes of cell lines derived from metastatic melanoma and non-small cell lung cancer, with less than 1% of these being non-synonymous mutations in coding regions [26, 27].

Most methods for detecting chromosomal or nucleotide changes score only clonal alterations and exclude changes that can be present in only a small fraction of cancer cells within a tumor. However, the cancer genome is more than simply a sum of its clonal mutations. A primary tumor is itself genomically heterogeneous, with the genome of each cancer/tumor cell having a unique mutational signature. This genetic variation within a cancer cell population reflects its evolution and, importantly, serves as a reservoir of genetic diversity from which resistance to therapy may arise. Analysis of disseminated single cancer cells from patients with minimal residual disease has shown that there is a high level of genomic heterogeneity within individual lesions, as well as between primary tumors and metastatic cells [29]. While some of the current sequencing technologies have the potential to detect subclones with frequencies as low as 1 in 1,000 [30], detecting rarer subclones by DNA sequencing requires the evolution of more accurate thermostable polymerases. This intra-specimen mutational diversity in different mutations within genomes of cancer cells from a single cancer/tumor can be a major contributor to the diverse spectrum of clinical phenotypes.

To identify all the genetic alterations present in an individual tumor, it is necessary to interrogate single DNA molecules from individual cancer cells. The accuracy of current deep sequencing methods is inadequate to detect random mutations in single-molecules lower than a frequency of 10⁻⁴. In accordance with aspects described herein, a Restriction Fragment Length Polymorphism (RFLP)/Polymerase Chain Reaction (PCR)-based assay has been developed to identify mutations in single molecules that render the DNA non-cleavable by restriction enzymes. Mutations are then quantified by amplification of single molecules with primers flanking the intact cut site using real-time PCR and confirmed by DNA sequencing [31]. This random mutation capture (RMC) assay permits the calculation of random mutation frequency of the cells that compose various tumor and normal tissues. In contrast to the low frequency of mutations in DNA from normal human tissues and cells, DNA from adjacent tumor tissues and cells was determined to exhibit a high frequency of single-base substitutions. The mean mutation frequency of DNA from six tumor samples was 2.2×10⁻⁶. Individual mutation frequencies ranged from 0.6×10⁻⁶ to 4.8×10⁻⁶, an average increase of greater than 200-fold compared with paired normal tissues. A dramatic increase in the frequency of random mutations has also been independently demonstrated in lung tumors with the RMC assay [32]. The diversity of nucleotide substitutions in tumor DNA is verified by DNA sequencing, and indicates that each substitution is present in only one or a few cells, and that DNA from individual tumor cells comprises more than 10,000 random mutations (i.e. a mutator phenotype).

### Emergence of Resistance to Targeted Chemotherapies.

As used herein, the term “mutator phenotype” refers to observable characteristics resulting from mutations in genes, e.g., mismatch repair genes or error-prone polymerases, that normally function in the maintenance of genetic stability. For example, in cancer, a mutator phenotype is manifested by increases in mutation rates of cancer cells, in which some of the mutations provide a selective growth advantage, allowing its progeny to proliferate and populate the tumor. In some embodiments, cancer cells with a mutator phenotype are manifested by an increase in their mutation rate of at least about 2-fold, at least about 5-fold, at least about 10-fold, at least about 25-fold, at least about 50-fold, at least about 100-fold, at least about 150-fold, or at least about 200-fold, as compared to the mutation rates of normal cells.

As a result of a mutator phenotype, tumors comprise individual cells with mutant genes that can render them resistant to therapy or therapies directed against the tumor. It should be noted that, even with this high mutational load, there can be very few cells within a given tumor with mutations rendering them resistant to multiple independent agents [33]. Thus, the presence of large numbers of random mutations in individual tumor cells allows for and sometimes necessitates simultaneous or sequential administration of multiple therapies. However, the simultaneous administration of multiple chemotherapeutic agents is often limited by toxicity issues.

The emergence of resistance to imatinib in patients with chronic myelogenous leukemia (CML) provides clear evidence for the involvement of random mutations in drug resistance. Imatinib, commercially known as Gleevec, is an exceptionally effective single agent that specifically targets the BRC-ABL1 protein kinase and has become the primary treatment for CML [34]. Resistance, however, develops in more than 30% of patients and is mediated by point mutations in the ATP binding site of the kinase in cancer cells [35]. This resistance can be overcome by administration of a second drug, e.g. Nilotinib, that targets a different site on the BRC-ABL1 protein kinase; however further resistance is mediated by the emergence of additional point mutations, which, when present in the same cell, confer resistance to both drugs [36]. Evidence indicates that many of these resistance mutations were present in bone marrow before the onset of therapy [35]. Mathematical models of resistance in CML indicate that most of the imatinib resistance occurring within 2 years of therapy can be accounted for by preexisting resistance if one assumes a mutation rate of 10-8/base per cell division [37, 38]. Additionally, prostate cancer metastases, analyzed after treatment with anti-androgens, have revealed a variety of mutations in the androgen receptor (AR) gene in cancer cells, which can alter promoter preferences, RNA and protein stability and ligand specificity [39]. Similar to the BCR-ABL1 resistance mutations in CML, these mutations in the androgen receptor gene are present at low frequency prior to selection with anti-androgens, thus indicating the importance of a mutator phenotype in cancer cells to the emergence of resistance to targeted therapies.

### Targeting the Mutator Phenotype

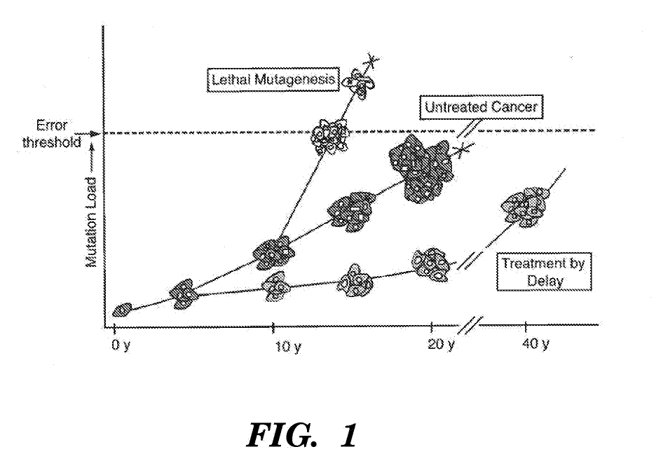

The large numbers of mutations present in human tumor cells indicate that the malignant phenotype is irreversible. As a result, chemotherapy of cancer has been directed at identifying compounds that selectively kill cancer cells or drive them into senescence. Success has been limited in large part due to the multiplicity of proteins that can functionally substitute for those targeted by specific chemotherapies, and by the subclonal and random mutations in cells within a tumor that provide a reservoir for the emergence of resistance to any given therapy. As shown herein, as a common feature of most cancers, the mutator phenotype can be an exploitable target for therapy. Targeting the increased rate of mutations in cancer cells can be accomplished by two alternative approaches as illustrated in FIG. 1. In some aspects, a method for treating cancer can reduce the incidence of mutation in precancerous diseases or during carcinogenesis to delay the onset or development of cancer. In other aspects, increasing the rate of mutation in cancer cells can incapacitate or kill malignant cells. The selection of the approach can be dependent, for example, on the rate and/or frequency of mutations in different tumors, and the state of cancer in an individual.

### Lethal Mutagenesis as an Approach to Chemotherapy of Advanced Cancers

Provided herein are novel methods of treating cancer in a subject by increasing the mutation rate in tumors to effect lethal mutagenesis. Accordingly, in one aspect, described herein are methods and compositions to induce an error catastrophe in cancer cells that can be used as an option for therapy of specific tumors.

In some embodiments of these aspects, tumors containing a population of tumor cells that have accumulated large numbers of random, independent mutations can be maintained similar to a viral quasispecies. The concept of a quasispecies has been applied to RNA-based viruses that mutate at high frequencies. RNA-based viruses have an exceptionally high mutation rate (1×10⁻⁴), which provides adaptability at the cost of viability; the vast majority of hepatitis C virus or HIV produced from an infected cell are unable to infect or reproduce, while the viable fraction of particles is marked by viral genomic and phenotypic heterogeneity [50]. Populations of virus that mutate at high frequencies are maintained as quasispecies, that are composed of a spectrum of viral genome mutants that can include rapid and slow propagating variants, defective viruses, as well as drug-resistant and -sensitive viruses [51-53]. Accordingly, in a viral quasispecies, vigorously replicating variants assure the survival of the entire population, while the poorly replicating variants provide genetic flexibility to facilitate survival within, and rapid adaptation to, a changing selective environment.

The human body is a hostile environment to tumor cells, which benefit from a suite of genomic mutations that permit abnormal cell divisions and can account for resistance to killing by chemotherapy. Strong selective pressure, such as the presence of anti-cancer agents or a robust immune response, favors cancer cells with a higher genomic mutation rate and greater phenotypic diversity. As in the case of HIV, cancer cells can have a limited tolerance for additional mutations, achieved by balancing flexibility with viability.

Accordingly, described herein are methods and compositions for increasing mutation rate or mutation frequency in the genome of cancer cells. The methods comprise contacting a cancer cell with one or more mutagenic compounds, where the one or more mutagenic compounds increase the mutation frequency of a cancer cell or progeny cell thereof by at least two-fold relative to the mutation frequency of the cancer cell or progeny cell thereof when not contacted by the one or more mutagenic compounds.

When the contacting is in vivo, the methods and compositions described herein for increasing mutation rate or mutation frequency in the genome of cancer cells can be employed in a subject for treatment of cancer. The methods of treating cancer in a subject in thereof include administering to a subject a pharmaceutically effective amount of a mutagenic compound as described herein, where the mutagenic compound increases the mutation rate of a cancer cell or progeny cell thereof. In some embodiments, the methods include administering to a subject a pharmaceutically effective amount of a mutagenic nucleoside analog, wherein the mutagenic nucleoside analog increases the mutation rate of a cancer cell or progeny cell thereof.

In some embodiments, the likelihood of normal and cancer cells or progeny cells thereof acquiring a dominant lethal genomic mutation can be equal per cell division. However, in other embodiments, the large number of acquired somatic alterations in cancer cells or progeny cells thereof can increase the likelihood of cancer cells or progeny cells thereof acquiring a synthetically lethal genomic mutation, as compared to the likelihood of normal cells acquiring a lethal genomic mutation. In such embodiments, an increase in the mutation rate in tumor cells is more detrimental than a similar increase in the mutation rate in normal cells.

As used herein, the phrase “therapeutically effective amount” means a sufficient amount of an active agent, e.g., a mutagenic compound, to treat a disorder, e.g., cancer, at a reasonable benefit/risk ratio applicable to any medical treatment. Accordingly, the term “therapeutically effective amount” refers to an amount of a mutagenic compound (e.g., mutagenic nucleoside) as described herein that is sufficient to effect a therapeutically significant reduction in a symptom or clinical marker associated with cancer, e.g., advanced cancer, when administered to a subject in need thereof. A therapeutically significant reduction in a symptom is, e.g. at least about 10%, at least about 20%, at least about 30%, at least about 40%, at least about 50%, at least about 60%, at least about 70%, at least about 80%, at least about 90% or more, in a measured parameter as compared to a control or non-treated subject. Measured or measurable parameters include clinically detectable markers of disease, for example, elevated or depressed levels of a tumor marker, as well as parameters related to a clinically accepted scale of symptoms for a disease or disorder, e.g., tumor growth. It will be understood, however, that the dosage of the compositions and formulations as described herein will be decided by the attending physician within the scope of sound medical judgment. The exact amount required will vary depending on factors such as the type of disease being treated.

With reference to the treatment of a subject with a cancer, the term “therapeutically effective amount” refers to the amount that is safe and sufficient to prevent or delay the development and further growth of a tumor or the spread of metastases in cancer patients. The therapeutically effective amount of a mutagenic compound can thus slow the course of cancer progression, slow or inhibit tumor growth, slow or inhibit tumor metastasis, slow or inhibit the establishment of secondary tumors at metastatic sites, or inhibit the formation of new tumor metastases.

The therapeutically effective amount of a mutagenic compound for treatment of cancer depends on a number of factors, which include, but are not limited to, the number of pre-existing mutations of the tumor, the type of tumor to be treated, the severity of the tumor, the drug resistance level of the tumor, the age and general condition of the subject, and the mode of administration. One of ordinary skill in the art can optimize the therapeutically effective amount or dosage accordingly. For example, the efficacy of a dosage of a mutagenic compound can be assessed in animal models of cancer and tumor, e.g., transplantation of human tumor cells into rodents such as mice or rats. When using an experimental animal model, efficacy of treatment is evidenced when a reduction in a symptom of the cancer, for example a reduction in the size of the tumor, or a slowing or cessation of tumor growth in treated, versus untreated animals.

In various embodiments, the therapeutically effective amount of the mutagenic compounds described herein (e.g., mutagenic nucleoside analogs), are sufficient to decrease a tumor size or delay the growth of a tumor, by at least about 10%, at least about 15%, at least about 20%, at least about 25%, at least about 30%, at least about 35%, at least about 40%, at least about 45%; at least about 50%, at least about 55%, at least about 60%, at least about 65%, at least about 70%, at least about 75%, at least about 80%, at least about 85%, or at least about 90%, at least about 95% or more, as compared to the tumor size in the absence of the administration. In some embodiments, the therapeutically effective amount of the mutagenic compounds described herein (e.g., mutagenic nucleoside analogs) are sufficient to decrease a tumor size or delay the growth of a tumor by at least about 50% or more, as compared to the tumor size in the absence of the administration. In some embodiments, the therapeutically effective amount of the mutagenic compounds described herein (e.g., mutagenic nucleoside analogs), are sufficient to inhibit or delay the growth rate of a tumor, by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, at least about 95% or more, as compared to the growth rate of a tumor in the absence of the administration. A tumor size can be determined and/or monitored in vivo by any imaging or other methods known to one of skill in the art, e.g., MRI or X-ray.

In various embodiments, the therapeutically effective amount of the mutagenic compound described herein (e.g., a mutagenic nucleoside analog) is sufficient to delay or inhibit the spread of metastases, e.g., by least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, at least about 95% or more, as compared to the degree of metastases in the absence of the administration. The degree of metastases can be determined, e.g., by the presence and the number of metastases arisen elsewhere in the body using imaging methods, such as chest X-ray for lung metastases or bone scan for bone metastases.

In some embodiments, a mutagenic compound described herein (e.g., a mutagenic nucleoside analog) or a composition comprising a mutagenic compound is administered to a subject in a dose ranging from about 0.01 mg/kg to about 1000 mg/kg, from about 0.1 mg/kg to about 500 mg/kg, or from about 0.1 mg/kg to about 250 mg/kg. In some embodiments, a mutagenic compound described herein (e.g., a mutagenic nucleoside analog) or a composition comprising a mutagenic compound can be administered to a subject in a nanomolar range to millimolar range of concentrations, depending upon the potency and efficacy of each such mutagenic compound. In some embodiments, a mutagenic compound described herein (e.g., a mutagenic nucleoside analog) can be administered in a concentration range from about 1 nM to about 1000 nM, from about 10 nM to about 750 nM or from about 100 nM to about 500 nM. In other embodiments, the concentration range of a mutagenic compound described herein (e.g., a mutagenic nucleoside analog) administered to a subject can vary from 1 μM to about 1000 μM, from about 10 μM to about 750 μM, or from about 100 μM to about 500 μM. In alternative embodiments, the concentration range of a mutagenic compound described herein (e.g., a mutagenic nucleoside analog) administered to a subject can vary from 0.1 mM to about 100 mM, from about 0.5 mM to about 50 mM, or from about 1 mM to about 10 mM.

In various embodiments, the mutagenic compounds described herein (e.g., mutagenic nucleoside analogs) can be administered to a subject orally or parenterally, e.g., via intravenous, intramuscular, intraperitoneal, intradermal, subcutaneous or intratumoral injection. Parenteral administration of the mutagenic compounds described herein can be performed by injection. In some embodiments, the mutagenic compounds described herein can be administered directly to a tumor, e.g., by surgery or long-needle injection, depending upon the location of the tumor.

In administering the mutagenic compounds described herein to a subject, dose amounts and administration schedules can be determined and optimized based upon a number of factors, e.g., tumor type, tumor condition, physical (e.g., age and weight) and health condition of the subject, potency and efficacy of the mutagenic compound, co-administration with another anti-cancer therapy, and route of administration. For example, the dose amount for oral administration can be higher than that for parenteral administration (e.g., by intravenous injection).

In some embodiments, the dose amount of the mutagenic compounds described herein (e.g., mutagenic nucleoside analogs) can be increased or decreased during the course of the treatment. By, way of example, during the course of the treatment, a higher dose of a mutagenic compound can be initially used to induce a greater degree of lethal mutagenesis in cancer cells, and the dose of the mutagenic compound can be lowered later to control the tumor growth. On the other hand, if the original dose amount of the mutagenic compound has no therapeutic effect on the tumor growth, a higher dose amount can be considered during subsequent treatments.

In other embodiments, the mutagenic compounds described herein (e.g., mutagenic nucleoside analogs) can be administered to a subject at any frequency, e.g., at least once a day, at least once every two days, at least once every three days, at least once a week or at least once every month. In some embodiments, the subject can be subjected to the mutagenic compound described herein daily. In other embodiments, the administration schedule of the mutagenic compound described herein (e.g., a mutagenic nucleoside analog) can vary during the course of the treatment. For example, the initial administration frequency of the mutagenic compound described herein to a subject can be more frequently, e.g., daily; and then the administration frequency can be decreased to, e.g., every other day or once a week, if the tumor responds to the treatment, e.g., a delay in tumor growth. In some embodiments, the subject can continuously receive the mutagenic compound described herein over a period of time, e.g., by a controlled-release drug delivery system. In such embodiments, the concentration of the mutagenic compound for a sustained release is generally lower, as compared to a dose given in a bolus.

In some embodiments, the mutagenic compounds described herein, e.g., mutagenic nucleoside analogs, can be administered to a subject in combination with any other anti-cancer therapies. Such “anti-cancer therapies” include, but are not limited to, additional mutagenic compounds, anti-cancer agents, surgical procedures to remove at least a part of the tumor mass, chemotherapy, radiation therapy (e.g., with gamma irradiation, X-rays, radioisotopes), and photodynamic therapy (e.g., with 5-aminolevulinic acid). There can be a delay of minutes, hours, days or weeks between administration of the mutagenic compounds described herein (e.g., mutagenic nucleoside analogs) and an additional therapy, such that the administration of the mutagenic compounds described herein can be administered before or after the other therapy. However, the administration of a mutagenic compound described herein to an individual can have inherent risks, for example, the risk of causing further tumors, that would preclude such action in some individuals. Thus, the administration of such mutagenic compounds should be considered only in cases in which the patient has an advanced cancer that is resistant to other therapies. In those situations, risks of treatment with a mutagenic compound can be outweighed by the immediate benefit of treating an existing tumor, which is otherwise essentially untreatable. This approach can be viewed as providing an option that will extend the productive life of an otherwise terminal patient beyond the timeframe otherwise available. Further, considerations that can mitigate the risk of promoting new tumorigenesis are disclosed elsewhere herein that can expand the range of indicated therapeutic application of the methods described herein.

In some embodiments, the mutagenic compounds described herein (e.g., mutagenic nucleoside analogs) can be administered to a subject in combination with at least one different active agent. The active agent can be administered to a subject prior to, concurrently with, or after the administration of the mutagenic compounds. Without limitations, the active agent can include, a small molecule, a peptide, a protein, a nucleic acid molecule, an antibody, an oligonucleotide, a nucleoside analog, a nucleoside mimic, a nucleotide analog, and a nucleotide mimic.

In some embodiments, the active agent is an anti-neoplastic chemotherapeutic agent or an anti-cancer chemotherapeutic agent. Examples of anti-neoplastic chemotherapeutic agents or anti-cancer chemotherapeutic agents include, but are not limited to, polyglutamic acid-paclitaxel; BMS-184476; Paclimer microspheres with encapsulated paclitaxel; taxane (IV) of Bayer; BMS-188797; epothilone B and analogs thereof including BMS-247550; ILX-651; N-[3-[(aminocarbonyl)amino]-4-methoxyphenyl]-2,3,4,5,6-pentafluorobenzenesulfonamide; T-900607; BAY 59-8862; T-138067; N,N-dimethyl-L-valyl-L-valyl-N-methyl-L-valyl-L-prolyl-N-(1,1-dimethylethyl)-L-prolinamide; benzoylphenylurea; trimetrexate glucuronate; 5-aza-2′-deoxycytidine; tocladesine; imatinib; PTK-787; BAY-439006; N-[4-[(3-chloro-4-fluorophenyl)amino]-7-[3-(4-morpholinyl)propoxy]-6-quinazolinyl]-2-propenamide; GW-572016; EKB-569; CP 609754; CI-1033; CCI-779; BMS-214662; (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile; cilengitide; bevacizumab; PK-412; IMC-1C11; 1-(2-chloroethyl)-2-[(methylamino)]carbonyl}-2-(methylsulfonyl)hydrazide; VNP-40101M; camptothecin glycoconjugate; liposome lurtotecan; gallium maltolate; N-[(3S,4E)-3-hydroxy-7-mercapto-1-oxo-4-heptenyl]-D-valyl-D-cysteinyl-(2Z)-2-amino-2-butenoyl-L-valine (4-1)-lactone cyclic (1-2) disulfide; buthionine sulfoximine; BMS-275291; phenylacetate; MS-275; chloroquinoxaline sulfonamide; INX-3280; phosphorothioate antisense oligonucleotide; GTI-2501; GTI-2040; K-ras protein vaccine; K-ras antisense oligonucleotide; MG-98; liposome C-raf antisense oligonucleotide; liposome raf-1 antisense oligonucleotide; SPD-424; Abarelix-depot; ERA-923; GTx-006; ILX 23-7553; 2B1 bispecific MAb; 3A1 MAb; SS1(dsFv)-PE38; chimeric TNT 1/B labeled with I-131; MAb Hum291; MEDI-507; HumaRad-HN; HumaRad-OV; MAb humanized CD3; Mylotarg; MAb-CTLA-4; cetuximab; BEC2; chimeric MAb 14.18; anti-transferrin receptor MAb; epratuzumab; MGS rCEA; INGN-241; CV-787; peripheral blood lymphocytes transduced with a gene encoding a chimeric T-cell receptor; BCI Immune Activator; Interferon-alpha gene therapy; Xcellerate; interleukin-2+staphylococcal enterotoxin B; NBI-3001; beta-alethine; APC-8020; interleukin-2/superantigen B gene combination; Melacine vaccine; SD/01; ALVAC B7.1 vaccine; APC-8024; GnRH Pharmaccine vaccine; rV-MUC-1; HPV 16 E6 and E7 peptide vaccine; allogeneic colon cancer vaccine; allogeneic glioma vaccine; autologous vaccine; VHL peptide vaccine; myeloma-derived idiotypic antigen vaccine; CaPVax; idiotype KLH lymphoma vaccine; LHRH immunotherapeutic (synthetic peptide vaccine); MAGE-12:170-178 peptide vaccine; MART-1 melanoma vaccine; MART-1 with gp100; rF-tyrosine vaccine; ESO-1:157-165 peptide vaccine; fowlpox-CEA(6D) tricom and vaccinia-CEA(6D) tricom vaccine; fowlpox gp100:ES 209-217 (2m) vaccine; RAS 5-17 peptide vaccine; proteinase-3 peptide vaccine; canarypox CEA; Helicobacter pylori vaccine; P53 and RAS vaccine; BAM-002; MedPulser in combination with bleomycin; lasofoxifene; Filmix; L-377202; T4N5 Liposome Lotion; Egr-1+TNF-alpha; aprepitant; skeletal targeted radiotherapy; combretastatin; CDC-501; taurolidine; Oramed; nystatin; Dynepo gene activated EPO; NC-100150; NC-100100; CDC-801; atrasentan; Aranesp; RK-0202; SB-251353; rasburicase; AFP-scan; Lymphoscan; ADL 8-2698; carboxypeptidase G2; metoclopromide nasal; dalteparin; MK-869; monomethyl arginine; repifermin; rH TPO; SR-29142; ancestin; CP-461; and Bexxar.

In some embodiments, the mutagenic compounds, e.g., mutagenic nucleosides, as described herein, are preferentially incorporated into tumor cells, because incorporation of the mutagenic compounds are more frequent in cancer cells, for example, due to pre-existing genomic mutations that widen the nucleotide binding pocket in DNA polymerase, or mutations that deregulate DNA repair processes. However, the mutagenic compounds (e.g., mutagenic nucleosides) described herein can be incorporated into DNA of non-malignant cells (e.g., benign tumor cells or non-cancerous cells), form mispairs at the time of DNA replication, and cause mutations. While not wishing to be bound or limited by theory, there can be four factors that mitigate the consequences of increasing mutations in normal or non-malignant cells. First, although a small increase in the frequency of random mutations in cancer cells can be sufficient to induce secondary tumors, mutations are much less frequent in normal or non-malignant cells, and a greater degree of random mutagenesis is required to induce carcinogenesis. Second, it takes many years from the time of exposure of an individual to a mutagen to the clinical appearance of a tumor. Third, normal or non-malignant cells can be more sensitive to the presence of mutagenic compounds and nucleotide pool imbalances than cancer cells, and can either repair or arrest with greater efficiency than cancer cells, minimizing any mutagenic consequences of treatment. Fourth, normal rapidly dividing cells, such as bone marrow and colonic epithelial cells, can be more sensitive to mutations accumulation than some slowly dividing cancer cells.

It is contemplated, without wishing to be bound or limited by theory, that normal cells can tolerate a mutagenic strategy by virtue of intact DNA repair and recognition capacities. Some recent efforts in the design of chemotherapeutic drugs have targeted DNA damaging agents to cancer cells that are sensitized to that type of damage, based either on the cellular phenotype, or on the effects of an inhibitor. For example, current clinical trials for glioblastoma are centered on poly (ADP ribose) polymerase (PARP-1) inhibitor and combined Temozolomide (TMZ)/radiation. By inhibiting PARP-1, cells are less able to repair mismatch repair-based DNA damage introduced by TMZ, and the radiation helps to convert damaged DNA into double-stranded breaks. Another example is the selective killing of aneuploid cancer cell lines by inhibiting the mitotic spindle checkpoint with siRNAs against Mps1 and BubR1 in combination with low dose Paclitaxel (taxol) treatment, which inhibits microtubule dynamics [56]. Preexisting aneuploidy appears to be necessary to use this strategy effectively, as similarly-treated immortalized diploid fibroblasts that maintained ploidy had no synergistic or additive susceptibility to taxol induced cell death when Mps1 expression was knocked down.

In some embodiments of the aspects described herein, expression of a mutator phenotype by a cancer cell can be used for diagnosis and therapy. Mapping mutated microsatellite sequences in a tissue, e.g., colon, using standard endoscopic biopsies has revealed a powerful predictive tool for pinpointing likely areas of malignancy in patients with inflammatory-associated diseases, e.g., ulcerative colitis, and can provide a meaningful marker for tracking the effectiveness of such therapy [20]. There is a need for diagnostics to identify the underlying molecular defect(s) behind the mutator phenotype in a given cancer or a cancer cell. A variety of studies have determined that a highly microsatellite instable (MSI-H) phenotype is associated with a more poorly differentiated cancer, but also with better overall prognosis than a microsatellite stable (MSS) phenotype [57-59]. In one embodiment, MSI-H cells, having acquired a large number of mutations and/or alterations, are more susceptible to the introduction of synthetic lethal mutations, such that the only recourse against this is to lose the MSI-H phenotype, and thus can be selected for use with the methods described herein. Additionally, the greater genomic instability in MSI-H cells can lead to a greater frequency of immunogenic epitopes in the affected cells. Accordingly, in some embodiments, enhancing mutagenesis, in addition to increasing the odds of introducing synthetic lethal mutations in a cell or cancer cell, can also be used to enhance the host immune response.

### Selection of Mutagenic Compounds for Lethal Mutagenesis

Since the initial discovery of 5-fluorouracil more than fifty years ago [54], a variety of nucleoside analogs have been used in the treatment of human cancers. Many of these have been selected based on termination of DNA chain elongation, or on inhibition of enzymes involved in deoxynucleoside metabolism such as thymidylate synthase and ribonucleotide reductase. The unscheduled termination of DNA replication by stalled replication forks leads to DNA breaks, cell cycle arrest, and potentially apoptosis [55]. Other agents that bind to DNA can also interact with free deoxynucleosides and alter their base pairing properties, or perturb nucleotide pools. Among these are alkylating agents used in the treatment of acute leukemias and glioblastomas, and ionizing radiation that induces DNA breaks.

As described herein, any mutagenic compound that increases the mutation rate or mutation frequency of a mammalian cell, such as a cancer cell, can be used for lethal mutagenesis. In some embodiments, a mutagenic compound that can be used for lethal mutagenesis increases the mutation rate or mutation frequency of a mammalian cell by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, at least about 100-fold, at least about 1000-fold or more, as compared to the mutation frequency of an untreated mammalian cell. In one embodiment, a mutagenic compound useful for lethal mutagenesis results in at least a 2-fold increase in the mutation frequency of a mammalian cell at its half maximal inhibitory concentration (IC50) for cell viability or lower, as compared to the mutation frequency of an untreated mammalian cell.

The term “mutation” is used broadly herein and refers, for example, to point mutations, missense mutations, silent mutations, frame shift mutations, nonsense mutations, insertions or deletions of nucleotides, loss-of-function mutations, gain-of-function mutations, and dominant negative mutations. In one embodiment, as used herein, the term “mutation”, means that a nucleotide sequence, such as the nucleotide sequence of a gene, is altered or refers to a state of the altered nucleic acid or the resulting amino acid sequence of the gene. For example, the term “mutation” herein refers to a change in the nucleotide sequence of a gene leading to a change in the resulting protein's function, such as a change in the exonuclease function of a polymerase. However, mutations described herein need not be limited to a change in a nucleotide sequence that encodes a protein. For example, mutations in non-coding regions such as regulatory regions can lead to loss or enhancement of function. The terms “mutation rate” and “mutation frequency” are used interchangeably herein, and refer to the absolute number of mutations per base per cell division. Depending upon the expression of a mutator phenotype, the mutation rate can range from about 10⁻⁸ mutations per base per cell division to about 10⁻⁶ mutations per base per cell division. In some embodiments, cancer cells can have a mutation rate of about 10⁻⁷ mutations per base per cell division, about 10⁻⁶ mutations per base per cell division or higher. As defined herein, the phrase “mutation frequency of a cancer cell” refers to number of mutations per base per cell division in the DNA of a cancer cell.

In some embodiments, the mutagenic compound for lethal mutagenesis can be a mutagenic nucleoside. Mutagenic nucleosides are an attractive class of lead molecule for lethal mutagenesis, and are designed to efficiently introduce mutations that are not rapidly repaired. As used herein, the term “mutagenic nucleoside” encompasses a nucleoside analog or a nucleoside mimic that can increase the mutation frequency of a mammalian cell, e.g., by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, at least about 100-fold, at least about 1000-fold or more, as compared to that mutation frequency of a mammalian cell in the absence of the mutagenic nucleoside. Methods for determining the mutation frequency of a mammalian cell are discussed later in detail. In one embodiment, a mutagenic nucleoside is incorporated by a polymerase. In another embodiment, a mutagenic nucleoside is not incorporated by a polymerase. In some embodiments, a mutagenic nucleoside can be a mutagenic RNA nucleoside or a mutagenic DNA nucleoside. In some embodiments, a mutagenic nucleoside is a mutagenic DNA nucleoside.

A “nucleoside” comprises a heterocyclic nitrogenous base, e.g., adenine, guanine, cytosine, uracil or thymine, joined to a ribose or deoxyribose sugar. Examples of nucleosides include adenosine (A), guanosine (G), uridine (U), thymidine (T) and cytidine (C). Sometimes, the term “deoxynucleoside” is used specifically to describe a nucleoside comprising a nitrogenous base joined to a deoxyribose sugar. Accordingly, the term “nucleoside” as used herein also encompasses the term “deoxynucleoside.” To denote a deoxynucleoside with a letter code, a prefix letter “d” is added to the letter code for a nucleoside, i.e., for example, dA is short for deoxy-adenosine. Throughout the specification, when referring to chemical modification of a nitrogenous base of a mutagenic nucleoside analog, the base numbering follows the IUPAC numbering system, which assigns an unique number to each atom of the nitrogenous base. Upon the addition of a phosphate group to the primary alcohol group located on the sugar of a nucleoside, the nucleoside becomes a nucleotide. Nucleosides or nucleoside analogs described herein, as used interchangeably herein, can be naturally occurring or synthetic.

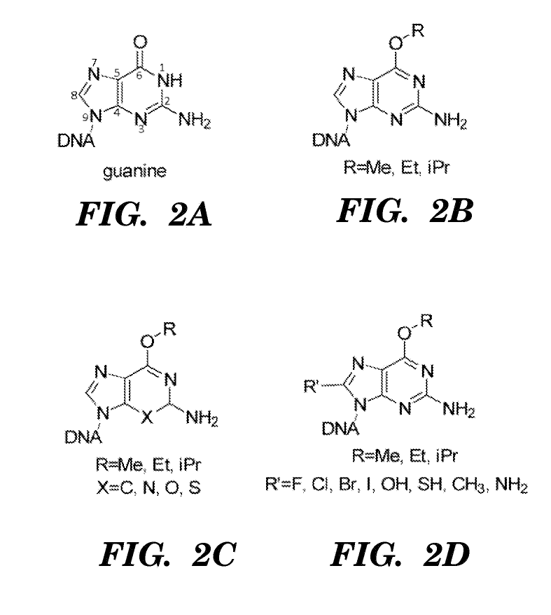

The term “analog” as used herein indicates a structural analog, i.e. a compound in which one or more atoms, functional groups, or substructures of the parent compound have been replaced with different atoms, groups, or substructures. The term “nucleoside analog” as used herein includes structural analogs of ribonucleosides and deoxyribonucleosides and the triphosphates thereof. They can be naturally occurring or non-naturally occurring, and be derived from natural sources or synthesized. In some embodiments, the nucleoside analogs, in reference with phosphorylation, can be considered as “nucleotide” analogs. A nucleoside “analog,” as the term is used herein, preferably retains structure sufficient to interact with one or more mammalian polymerase enzymes in a manner that either permits incorporation of the analog into the genome of the cell, or that influences the ability and/or selectivity of the polymerase to incorporate the analog or other nucleoside into genetic material. Thus, the term “analog” as applied to nucleosides refers in one embodiment, to a structurally different nucleoside that is incorporated by a polymerase into the DNA of the cell, and in another embodiment, to a structurally different nucleoside that, while not necessarily incorporated by a polymerase, influences (e.g., decreases by at least 5%, preferably at least 10%, at least 20%, at least 30%, at least 40%, at least 50%, at least 60%, at least 70%, at least 80%, at least 90% or more, e.g., at least 2-fold, at least 5-fold, at least 10-fold or more) the fidelity of polymerase for incorporation of the correct nucleoside. As non-limiting examples shown in FIGS. 2A-2D, various deoxyguanosine analogs can be generated from a deoxyguanosine by replacing one or more constituents (atoms) with a different atom or a functional group.

The terms “nucleoside mimic” and “nucleoside mimetics,” as used interchangeably herein, refer to a molecule that mimics the shape, function or other characteristics of a nucleoside, while lacking one or more structural determinants of the nucleoside as defined herein (e.g., lacking a sugar or purine or pyrimidine nitrogenous base). For example, the difluorotoluene isostere is considered as a shape mimic of a nucleoside thymidine (dT) in the crystalline state and in solution. The chemical modification involves replacing oxygen of the dT nitrogenous base with fluorine and nitrogen of the dT nitrogenous base with carbon, and keeping aromaticity intact. See Kool, 35 Acc. Chem. Res. 936 (2002). In some embodiments, the nucleoside mimics, in reference with phosphorylation, can be considered as “nucleotide” mimics. In some embodiments, the nucleoside mimics can be shape mimics of nucleoside analogs described herein.

Examples of nucleoside mimics or nucleoside mimetics include, but are not limited to, acyclic nucleosides, e.g., acyclovir (prodrug: valaciclovir), ganciclovir (prodrug: valganciclovir), and penciclovir (prodrug: famciclovir); acyclic nucleoside phosphonates, e.g., cidofovir, adefovir, and tenofovir; hydrazono acyclic nucleosides; allylic/acyclic adenosine nucleoside triphosphate, e.g., 4-adeninyl-2-(hydroxymethyl)-2-butenyl triphosphate; HPMP (3-hydroxy-2-phosphonomethoxypropyl) and PME (2-phosphonomethoxyethyl) derivatives, 6-[2-(phosphonomethoxy)alkoxy]-2,4-diaminopyrimidine (DAPy) derivatives, and HPMP derivatives containing a 5-azacytosine moiety; 5-substituted acyclic pyrimidine nucleosides, e.g., 5-(1-azido-2-haloethyl)uracil), 1-[(2-hydroxyethoxy)methyl], 1-[(2-hydroxy-1-(hydroxymethyl)ethoxy)methyl], and 1-[4-hydroxy-3-(hydroxymethyl)-1-butyl]; 3′,5′-dideoxy-5′-C-phosphonomethyl nucleosides; 2-phosphonomethyl-1,3-dioxolane nucleosides containing appropriately linked pyrimidine and purine bases at C-4 position; sialyl nucleoside mimetics based on D-fructose, e.g., 1-(methyl 3,4-anhydro-6-thio-6-[2′-(sodium butyrate)]-α-D-tagatofuranoside)-uracil; 6-(β-D-ribofuranosyl)-2-alkyl/aryl-6H-imidazo[1,2-c]pyrimidin-5-one and 6-(β-D-ribofuranosyl)-5-oxo-5,6-dihydro-imidazo[1,2-c]pyrimidine-2-carbonitrile, and 2,3,5,6-tetrahydro-imidazo[1,2-c]pyrimidine-2-carbonitrile nucleosides. In addition, the 5′ monophosphate mimics disclosed in U.S. Patent Application No.: US 20040023901, e.g., 5-ethnynl-1-(1-D-ribofuranosyl)imidazole-4-carboxamide 5′ phosphorothioate, can also be used in compositions and methods described herein. See De Clercq E., 16 Clinical Microbiology Reviews, 569 (2003); Holy A. Current Protocols in Nucleic Acid Chemistry. Chapter 14; Unit 14.2 (2005); Mohammad N. et al., 6 Beilstein J. Org. Chem. 49 (2010); Ilya L. et al., 51 Antimicrobial Agents and Chemotherapy 2268 (2007); Guo H. et al., 75 J. Org. Chem. 3863 (2010); Martinez C. I. et al., 27 Nucleic Acids Res. 127 (1999); Rakesh K. et al., 44 J. Med. Chem. 4225 (2001). Ioannidis P. et al., 45 Acta Chem Scand. 746 (1991); Bednarski K. et al., 5 Bioorganic &Medicinal Chemistry Letters 1741 (1995); Simons C. Nucleoside mimetics: their chemistry and biological properties. CRC Press (2001); Grice I. D. et al., 16 Tetrahedron Asymmetry 1425 (2005); Vorbrüggen U. et al., Handbook of Nucleoside Synthesis (Organic Reactions). Wiley-Interscience (2001); Kifli N et al., 12 Bioorg Med Chem. 4245 (2004). Mutagenic nucleoside mimics are preferred for the methods described herein. Those listed above or known in the art that have one or more of the criteria listed below for mutagenic nucleoside analogs are contemplated for use in the methods described herein.

Mutagenic nucleosides or mutagenic nucleoside analogs are used in preference to agents that bind to DNA, in order to minimize damage to other cellular macromolecules (Rechkoblit O et al., 17 Nat Struct Mol Biol 379 (2010); Upton et al., 19 Chem Res Toxicol 960 (2006)). Factors to be considered for selection of an effective mutagenic nucleoside analog as a candidate for lethal mutagenesis include at least one of the following factors: (1) the mutagenic nucleoside analogs are stable and water soluble; (2) while incorporation of a nucleoside analog is not absolutely required for it to be mutagenic, one factor in selecting mutagenic nucleosides is that they harbor small substituents on the bases so that they can be easily incorporated by DNA polymerases, such as a methyl group, a ethyl group, or a halogen atom such as fluorine or chlorine. These substituents preferably favor the formation of hydrogen bonds with non-complementary bases at high frequency. (3) The mutagenic nucleoside analogs readily enter human cells and can be converted to nucleoside triphosphates by cellular nucleoside/nucleotide kinases or phosphotransferases. (4) The resulting deoxynucleoside triphosphate can be incorporated into DNA by the major replicating DNA polymerases, Pol-δ or Pol-ε, even in competition with the corresponding unaltered nucleoside triphosphate. (5) The mutagenic nucleoside analogs are resistant to detection or excision by a DNA repair process, e.g., BER or MMR processes, or are resistant to sanitization while in the nucleotide pool, i.e. the mutagenic nucleoside analogs are resistant to the removal from the DNA by base excision repair or from the nucleotide pool by the nucleoside sanitization enzyme. (6) The mutagenic nucleoside analogs preferably mispair at high frequency during replication, leading to progressive accumulation of mutations in a target cancer cell. The accumulation of these analogs can be augmented in certain cancers where the mutator phenotype results from mutations in replicative DNA polymerases that decrease base selection, or due to dysregulation of low-fidelity specialized DNA polymerases. In some embodiments, mutagenic nucleoside analogs can interact with DNA polymerase or RNA polymerase. Mutagenic nucleoside analogs that meet at least one of these criteria can encompass both natural purines and pyrimidines as well as those with novel chemical structures (e.g., nucleoside mimics). In some embodiments, the nucleosides or nucleoside analogs can have at least two of the characteristics described above. In further embodiments, the nucleosides or nucleoside analogs can have at least three, at least four, at least five, or even all six of the characteristics described above.

In some embodiments, a mutagenic nucleoside analog comprises a chemical modification of a nucleoside with a nitrogenous base selected from the group consisting of: adenine, guanine, thymine, uracil and cytosine. In various embodiments, the chemical modifications generally occur in the nitrogenous base. Examples of such chemical modifications to a nitrogenous base can include, but are not limited to: (1) replacement of a N atom with any atom selected from the group consisting of C, O and S; (2) replacement of a ketone group (═O) with a —OR¹ group, where R¹ can be a H, C₁-C₄ alkyl, C₂-C₄ alkenyl, C₂-C₄ alkynyl, C₁-C₄ haloalkyl, C₁-C₄ heteroalkyl, heteroaryl, or aryl, wherein the alkyl, alkenyl, alkynyl, haloalkyl, heteroalkyl, heteroaryl, and aryl can be optionally substituted with halogen, C₁-C₃ alkyl, C₁-C₃ haloalkyl, C₁-C₃ heteroalkyl or C₁-C₃ alkoxy; (3) replacement of a H atom attached to a C atom of a double bond with a —R² group, wherein R² can be a halogen, —OH, —SH, alkyl, and —NH₂; and (4) any combination thereof. In some embodiments, a double bond can be removed to accommodate any chemical modification.

Exemplary nucleoside analogs or derivatives thereof that can be used as mutagenic compounds for lethal mutagenesis in the compositions and methods described herein include, but are not limited to, O⁴-methyl thymidine, O⁴-ethyl thymidine, 8-hydroxy deoxyguanosine, etheno-dG, etheno-dA, etheno-dC, O⁶-methyl dG, O⁶-ethyl dG, 5-OH-methyl cytidine, 2-Amino-9-(β-D-2′-deoxyribfuranosyl)purine, 3-(2′-Deoxy-β-D-2-ribofuranosyl)pyrido[2,3-d]pyrimidine-2,7(8H)-dione (dF), and 2′-Deoxyinosine, each of which is further described below:

O⁴-methyl thymidine: This nucleoside analog mispairs with dG at a frequency of approximately 25% (Dosanjh, M. K., Biochemistry) There is no known mechanism for the repair or removal of this analog in animal cells (Brent, T. P., Proc. Natl. Acad. Sci. 85: 1759, 1988). A nucleotide analog, O⁴-methyl thymidine triphosphate, has been shown to be incorporated into DNA by DNA polymerases (Singer, B., et al., Proc. Natl. Acad. Sci. 80: 4884, 1983);

O⁴-ethyl thymidine: This nucleoside analog mispairs with dG at a frequency of approximately 25% (Dosanjh, M. K., Biochemistry). There is no known mechanism for the repair or removal of this analog in animal cells (Brent, T. P., Proc. Natl. Acad. Sci. 85: 1759, 1988). A nucleotide analog, O⁴-ethyl thymidine triphosphate, has been shown to be incorporated into DNA by DNA polymerases (Singer, B., et al., Proc. Natl. Acad. Sci. 80: 4884, 1983).

8-hydroxy deoxyguanosine: This nucleoside analog, also referred to as 8-oxo-deoxyguanosine, can be used for the therapy of cancer. For example, this nucleoside analog can be used for enhancing the mutagenesis of the cancer based on the established high frequency of mispairing by this analog, when introduced into DNA (Cheng, K. C., et al., J. Biol. Chem., 267: 166, 1992) or when present with a DNA template (Wood, M. L., et. al., Biochem., 29: 7024, 1990; Shibutani, S. et al., Nature, 349: 341, 1991). This analog has not been previously used or suggested for the treatment of cancer by any means or for introducing mutations in cellular DNA. The mutagenic properties of this analog can be documented by the 1000-fold increase in mutagenesis in E. coli that lack a mechanism for the repair of 8-OH-dG (Michaels et al., Proc. Natl. Acad Sci., 89: 7022, 1992). The ability of HIV reverse transcriptase to incorporate 8-OH-dG has been demonstrated (Cheng, K. C. et. al., J. Biol. chem., 267: 166, 1992)). 8-OH-dG in double-stranded genomic DNA is excised by the FAPY glycosylase in E. coli (Chung, M. H., et al., Mutat. Res., 254, 1, 1991) and there is substantial evidence for the presence of a similar activity in animal cells. A number of studies have established the mutagenic potency of 8-oxo-dG in DNA (see Cheng, K. C. et. al., J. Biol. chem., 267: 166, 1992) using site-specific mutagenesis. Studies using DNA containing 8-OH-dG at a specific location indicate that the frequency of mispairing in E. coli varies from 0.5 to 50% presumably depend on the efficiency of repair of the DNA containing such modification. The non-repaired nucleoside analog can base pair with deoxyadenosine at a high frequency.

Etheno-dG, Etheno-dA, or Etheno-dC: Each of these nucleoside analogs comprise additional ring structures that interfere with base-pairing and each forms non-complementary base-pairs at a frequency of approximately 10%. N²,3-etheno dG forms predominantly G->A transitions and the triphosphate analog is incorporated by DNA polymerase-α (Cheng, et al., Proc. Natl. Acad. Sci. 88: 9974, 1991). N³,4-etheno dC forms both C->T and C->A substitutions, while 1,N6-etheno dA forms A->G and A->C substitutions. A human protein has been identified that binds specifically to a single 1, N⁶-ethenoadenine in DNA and it has been suggested that this protein can guide a repair nuclease to the site occupied by this analog (Rydberg, B., et al., Proc. Natl. Acad. Sci. 88: 6839, 1991; Ryberg, B., et al., Canc. Res. 52: 1377, 1992)). In other studies, there is evidence that the binding protein is identical to 3-methyladenine glycosylase (Singer, B., Proc. Natl. Acad. Sci. 89: 9386, 1992). Alternatively, the etheno analogs described herein can be subjected to repair by a human endonuclease analogous to the uvrABC complexes that have been extensively characterized in bacteria.

O⁶-methyl dG or O⁶-ethyl dG: These nucleoside analogs have been shown to base-pair with thymidine at a frequency of 19% and 11%, respectively, compared to that of correct base-pairing with cytidine in human cells deficient in O⁶-methyl transferase (Ellison, K. S. Proc. Natl. Acad. Sci., 86: 8620, 1989). Individuals are known to exhibit marked differences in the repair or removal of these analogs, and those with decreased repair are referred to as exhibiting a mer⁻ or a mex⁻ phenotype. Methods are known in the art to deplete the level of methyl transferase based on the fact that the enzyme is subjected to suicide inhibition. In some embodiments, any method known to a skilled artisan for depletion of methyl transferase can be utilized in a combined protocol with the administration of a mutagenic analog for the treatment of cancer, if individuals have a high level of methyl transferase enzyme or activity.

5-OH-methyl cytidine: This nucleoside analog is reported to be mutagenic and forms non-complementary base-pairs with thymidine.

2-Amino-9-(β-D-2′-deoxyribfuranosyl)purine: The deoxynucleoside of 2-Aminopurine (2AP), can cause both transition and frame shift mutations in Escherichia coli. Without wishing to be bound by theory, the analog to be used in the methods and compositions described herein can cause mutations by two mechanisms: directly, by mispairing with cytosine, and indirectly, by saturation of mismatch repair [64, 65].

3-(2′-Deoxy-β-D-2-ribofuranosyl)pyrido[2,3-d]pyrimidine-2,7(8H)-dione (abbreviated as dF): When incorporated into oligonucleotides, dF was found to pair selectively with G and A in double helical DNA (G>A>>C or T), resulting in higher and lower melting temperatures, respectively, than the corresponding C-G and T-A pairs [66]. Not to be bound by theory, in one embodiment, dF can form a Watson-Crick base pair with G much as C does. In another embodiment, dF can, via a backbone shift, form a wobble base pair with A. Alternatively, dF can form a hydrogen bond with A via an alternate tautomer as shown [67,68]. In triple helices, dF pairs well with dA [67].

2′-Deoxyinosine: Universal bases can pair indiscriminately with any other base and can thus be mutagenic [69]. In addition, by altering the balanced supply of the precursors of DNA synthesis, i.e. the deoxyribonucleoside triphosphates (dATP, dCTP, dGTP and dTTP), 2′ deoxyinosine has been shown to have dramatic genetic consequences for mammalian cells including the induction of mutations [70].

In some embodiments, the nitrogenous base of a nucleoside can be replaced with a cyclic hydrocarbon, a heterocycle, or a combination thereof. In such embodiments, the cyclic hydrocarbon or heterocycle replacement can range in size from monocyclic to tetracyclic (e.g., benzene, naphthalene, phenanthrene and pyrene). See Kool 35 Acc. Chem. Res. 936 (2002), in which the molecules described therein can also be utilized for the compositions and methods described herein.

In some embodiments, the mutagenic nucleoside analogs described herein can be used as core compounds for the synthesis of nucleoside triphosphate derivatives using selective chemical modifications. In some embodiments, mutagenic nucleoside derivatives are prepared as their mono-, di- or triphosphates. The phosphorylation of the mutagenic nucleoside analogs is typically carried out by enzymatic methods, chemical methods or combinations of enzymatic and chemical methods. Enzymatic phosphorylation can be carried out using a crude source of wheat shoot extract as a source of phosphotransferase activity (see Giziewicz, et al., Acta. Biocim. Pol. 22:87-98 (1975); and Sugar, IN MOLECULAR ASPECTS OF CHEMOTHERAPY, Springer-Verlag, 1991, pp. 240-270). Phosphorylation can also be carried out using Herpes virus thymidine kinase or mutants thereof as described by Black, et al., Biochemistry 32:11618-11626 (1993). Herpes thymidine kinase has a wide substrate specificity and phosphorylates a wide variety of nucleoside analogs. Purification of the resultant monophosphates can be accomplished using, for example, HPLC.

Further conversion of the monophosphate derivatives to the triphosphates can typically use chemical methods such as those described in Hoard, et al., J. Am. Chem. Soc. 87:1785-1788 (1965).

Deoxynucleoside triphosphate derivatives of the mutagenic nucleoside analogs described herein can be chemically modified to obtain a series of analogs with different chemical groups on either the base or sugar moieties. One of ordinary skill in the art will readily recognize procedures for the chemical modification of these analogs. Selected analogs can be subjected to specified chemical modifications to introduce bulky substituents at different positions. After chemical modification, the analogs can be evaluated by DNA polymerases. Substitution of nucleoside triphosphate analog for the complementary nucleotide can be assessed by any method known in the art, e.g., chain extension by gel electrophoresis (for example, see Preston B D, Poiesz B J, Loeb L A. Fidelity of HIV-1 reverse transcriptase. Science. 1988 Nov. 25; 242(4882): 1168-71).

In some preferred embodiments, the modification of the specified mutagenic nucleoside analogs is limited in order to avoid the introduction of bulky groups on the analogs that already have a substituent for miscoding, as described in, for example Preston B D, Poiesz B J, Loeb L A. Fidelity of HIV-1 reverse transcriptase. Science. 1988 Nov. 25; 242(4882):1168-71, the contents of which are herein incorporated by reference in their entireties. In such embodiments, by avoiding introduction of bulky groups, inhibition of replication is avoided, and the modified mutagenic nucleoside analog can continue to be incorporated into a cell's genome.

It will be understood in the art that mutagenic nucleosides and analogs thereof can be prepared as racemic mixtures or as enantiospecific compositions which have a prevalence of one or another enantiomeric species (typically greater than about 70% prevalence, more preferably greater than about 90%, still more preferably greater than about 98%). For example, one can obtain or synthesize enanitio-specific nucleoside analogs that are predominantly in an unnatural or L-enantiomeric configuration (e.g., by incorporating an L-ribose in place of a D-ribose). In some embodiments, enantio-specific nucleoside compositions can be employed to further optimize efficacy (e.g., activity and/or specificity) and minimize toxicity.

In some embodiments, enantio-specific mutagenic nucleoside analogs (and/or other structural variations) can also be employed to influence the intracellular localization of analogs. An enantio-specific or other variant of mutagenic nucleoside analog can be more efficiently processed by cellular machinery involved in the uptake or intracellular localization of such analogs. For example, one can employ mutagenic nucleoside analogs which are preferentially taken up by (and/or localized in the cytosol of) a targeted mammalian cell. In some embodiments, such structural variations can also be used to influence the efficiency at which an analog is processed by an enzyme—such as cellular kinases capable of converting a nucleoside to a nucleoside phosphate prior to incorporation, or cellular degradative enzymes such as phosphatases.

The production of libraries containing random chemical substituents using the mutagenic nucleoside analogs described herein as starting compounds is also described herein. In some aspects, mutagenic deoxynucleoside analogs are used as core compounds for the synthesis of chemically diverse libraries, e.g., by combinatorial synthesis methods. Individual analogs can be subjected to reactions that chemically protect the miscoding substituents using standard methods of nucleotide chemistry. The protected analogs can be subjected to a series of chemical reactions involving but not limited to the following: dehydration, irradiation, oxidation, reduction, alkylations and permutations thereof. Thereafter, the resultant mixture of diverse products comprising a library of greater the five million and less than one million chemical species can be subjected to deprotection reactions.

Further embodiments also provide for the production of nucleoside derivatives of the miscoding libraries described herein by incubation of the miscoding libraries with extracts of human cells, including, but not limited to, red blood cells or other cell types that contain high levels of nucleoside monophosphate kinases. The reaction can be supplemented by Mg²⁺, ATP and/or an ATP generating system as well as other chemicals routinely used in kinase reactions for the phosphorylation of nucleosides. In some embodiments, human cell extracts are used as such extracts permit phosphorylation by endogenous kinases that are present in these cells. The resultant nucleotides can be separated as a group, using, for example, ion exchange resins by any method known in the art, e.g., high pressure liquid chromatography. In some embodiments, the resultant nucleosides can be administered to a subject in need thereof, e.g., to increase mutation frequency of at least one cell such as a cancer cell. However, as nucleotides that are generally charged cannot easily cross cell membranes, in some embodiments, the respective nucleosides can be administered instead. In those embodiments, the nucleosides can be converted into nucleotides inside the cells, e.g., by phosphorylation with an endogenous kinase. Alternatively, modifications that enhance the ability to cross cell membranes can be employed with nucleotides.

In some embodiments, candidate mutagenic deoxynucleoside triphosphate analogs can be separated and identified based on the mutation frequency of a cancer cell after contacting or treatment. In other embodiments, the candidate mutagenic deoxynucleoside triphosphate analogs can be separated and identified based on incorporation of the analogs into DNA by human DNA polymerases. A library of nucleoside triphosphates produced from the mutagenic nucleoside analogs and/or mutagenic deoxynucleoside analogs described herein and combining 2 to 100,000 different molecular species can be assessed in reaction with a DNA polymerase and three other normal deoxynucleoside triphosphates. The product of the reactions can be separated from the template by, for example, denaturing gel electrophoresis. The candidate mutagenic nucleoside analog that is incorporated into the product can be released by chemical and/or enzymatic digestion to yield the free base or the nucleoside. Its identity can then be determined by, for example, mass spectroscopy.

The resultant mutagenic nucleoside analogs can also be used as a core starting material for synthesis of second-generation candidate mutagenic nucleoside analogs, which can be subjected to various methods described herein to measure potential improvements: either incorporation into DNA by a human polymerase or the production of enhanced mutagenesis of the genome of a cancer cell after exposing human cancer cells in culture to the products of the reactions.

By way of example, the biochemical steps described herein are solely used to illustrate one of the feasible methods to generate candidate mutagenic nucleoside analogs and/or establish the identity of candidate mutagenic nucleoside analogs. Any other methods known to a skilled artisan can also be used for the purposes described herein. Nucleotides can be evaluated for their potential to miscode in vitro using assays that have been established to measure the fidelity of DNA polymerases (Fry, M., and Loeb, L. A., Animal Cell DNA Polymerases, 1986). Once identified, the corresponding free base and nucleoside can be synthesized in large amounts by standard procedures recognized in the art.

To identify potential mutagenic compounds (e.g., mutagenic nucleoside analogs) for treatment of cancer, an in vitro screening of candidate mutagenic compounds using cancer cells can be performed. By way of illustration, the in vitro screening can include determining the highest concentration of each candidate mutagenic compound at which no apparent cell toxicity is observed; and measuring the resultant accumulation of mutations in nuclear and mitochondrial DNAs. In some embodiments, the in vitro screening can further include monitoring cells for incorporation of the candidate mutagenic compound (e.g., candidate mutagenic nucleoside analog) into DNA or RNA and induction of mutagenesis. Cancer cells from any specific tissues can be used for in vitro screening assay. Exemplary cancer from which cells can be used include, but are not limited to, colon cancer, small cell lung cancer, prostate cancer, breast cancer, pancreatic cancer, and liver cancer.

To determine the highest concentration of each candidate mutagenic compound at which no apparent cell toxicity is observed, mammalian cells (e.g., mammalian cancer cells and mammalian non-cancerous cells) can be cultured in the presence of increasing concentrations of each candidate mutagenic compound for a period of time, e.g., at least 12 hours, at least 24 hours, at least 2 days, or at least 3 days, and then the cell viability at each candidate mutagenic compound concentration can be determined by various well-established methods, e.g., crystal violet staining, WST-1 assay (e.g., from ROCHE), MTT assay, LIVE/DEAD® viability assay (from Invitrogen), or Trypan blue exclusion assay. In some embodiments, the cell viability of cancer cells that are proficient in mismatch repair can be compared to that of cancer cells that are deficient in mismatch repair, in order to identify candidate mutagenic compounds (e.g., mutagenic nucleoside analogs) that induce cell death due to DNA or RNA mispairing and mutation. For example, as shown in the Examples (Table 2), some nucleoside analogs, e.g., O⁴-methyl-2′-deoxynthymidine, show similar toxicity in cancer cells regardless of their proficiency in mismatch repair. This can indicate that those nucleoside analogs are not mutagenic.

Without wishing to be bound by theory, as normal cells or non-cancerous cells accumulate fewer mutations than cancer cells, cancer cells should more readily reach the threshold of mutations, above which the cells are no longer viable, than normal cells do. Accordingly, in some embodiments, it is desirable for a mutagenic compound described herein (e.g., a mutagenic nucleoside analog) to have a minimal effect on the cell viability of normal cells, e.g., non-cancerous cells. In some embodiments, the reduction in the viability of cancer cells treated with a specified concentration of the mutagenic compound (e.g., mutagenic nucleoside analog) can be greater than that of normal cells treated with the same, e.g., by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, at least about 100-fold or more. In one embodiment, the mutagenic compound (e.g., mutagenic nucleoside analog) can have a comparable effect on cell viability of normal and cancer cells.

In accordance with the methods described herein, administration of a mutagenic compound (e.g., mutagenic nucleoside analog) can increase the mutation frequency of one or more cells, e.g., cancer cells. In various embodiments, the mutagenic nucleoside analogs useful for lethal mutagenesis can increase the mutation frequency of one or more cells, e.g., cancer cells, by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, at least about 100-fold, at least about 1000-fold or more, as compared to the mutation frequency of cancer cells cultured in the absence of the mutagenic nucleoside analogs. In particular embodiments, the mutagenic nucleoside analog can induce such mutation frequency of cancer cells when the cancer cells are treated with the mutagenic nucleoside analog at its half maximal inhibitory concentration (IC50) for cell viability or lower. In one embodiment, treatment of cancer cells with a mutagenic nucleoside analog at IC50 results in at least about 2-fold increase in mutation frequency of cancer cells. The mutation frequency of a cell in response to a mutagenic nucleoside analog can be identified by any mutation assay known in the art, e.g., PIG-A gene mutation assay, random mutation capture assay, hypoxanthine phosphoribosyltransferase (HPRT) mutagenesis assay or a combination thereof. See, for example, Araten et al., 108 Blood 734 (2006); Vermulsta et al., 46 Methods 263 (2008); Aidoo et al., 387 Mutat Res. 69 (1997), the contents of which are herein incorporated by reference in their entireties.

In another embodiment, the mutation frequency of a candidate mutagenic compound described herein (e.g., candidate mutagenic nucleoside analog) can be determined by its incorporation into the catalytic site of a human polymerase (or one of its homologs, e.g., yeast). For example, DNA polymerase can be isolated from the cells after treatment with a candidate mutagenic compound described herein, and then subjected to methods for determination of crystal structures, e.g., X-ray crystallography. Any methods known to a skilled artisan can also be used to determine a crystal structure of DNA polymerase with a nucleoside analog incorporated into an associated DNA primer. In other embodiments, computational methods for structural analysis can be used to predict the “fit” of a mutagenic compound into the catalytic site of a human polymerase or one of its homologs, e.g., yeast.

In some embodiments, the incorporation of a candidate mutagenic compound such as a candidate mutagenic nucleoside analog into DNA is not required for mutagenesis. For example, the presence of the candidate mutagenic compound can imbalance the nucleotide pool of the cell. In such embodiments, incorporation of the candidate mutagenic compound into DNA is not evaluated for induction of mutagenesis.

In some embodiments, the candidate mutagenic compounds useful for the treatment of cancer results in an increase in the mutation frequency of a mammalian cell. The mammalian cells selected for mutagenesis screening are cells associated with a disease or disorder of interest. By way of example, to identify a mutagenic nucleoside analog for treatment of human colon cancer, cells or tissues from human colon tumors are preferable. However, for a preliminary screening, cells from other animal models, such as a rodent, can also be used, followed by verification with human cells. The accumulation of mutations in nuclear (genomic) and mitochondrial DNAs resulting from treatment with a mutagenic compound can be measured by any mutation method known in the art, e.g., PIG-A gene mutation assay, random mutation capture assay or HPRT mutagenesis assay.

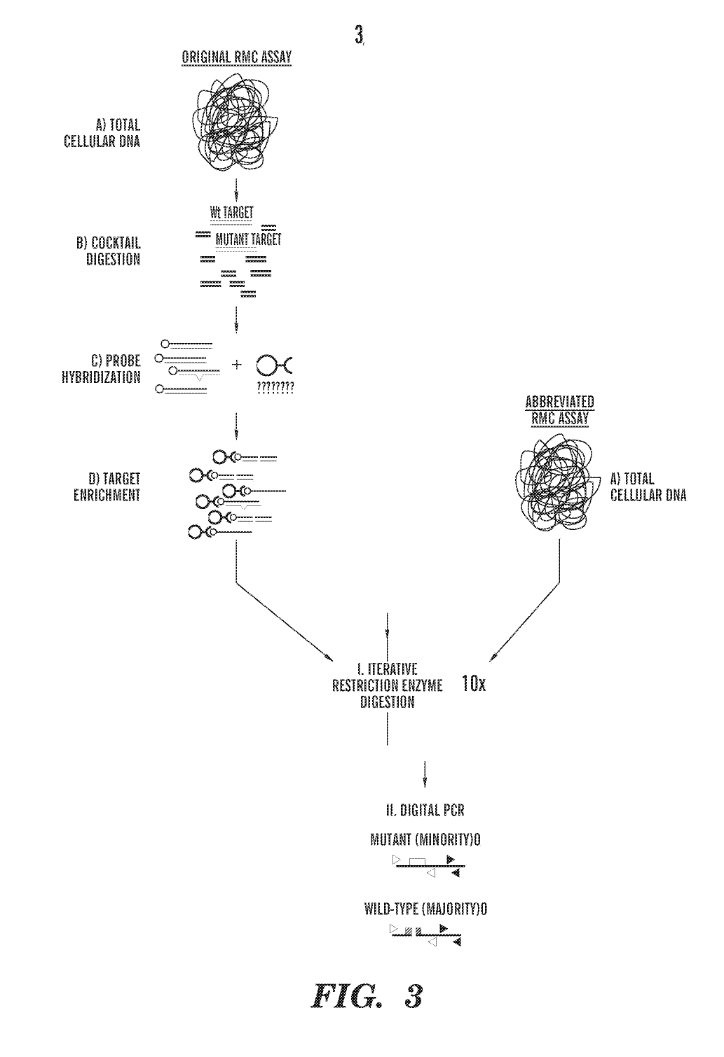

In one embodiment, a random mutation capture (RMC) assay is modified to measure the frequency of mis-incorporation in nuclear and mitochondrial DNAs, as shown in FIG. 3. For example, the amount of DNA to be digested by restriction enzyme is limited such that a series of target enrichment steps, as required in the original RMC assay, can be omitted. The omission of enrichments steps enables multiple sequences to be interrogated simultaneously and reduces the overall amount of DNA required to perform the assay.

The mammalian cells, e.g., mammalian cancer cells, can be cultured in the presence of a candidate mutagenic compound as described herein (e.g., a candidate mutagenic nucleoside analog) for a period of time with multiple serial passages, e.g., at least 25 serial passages, at least 50 serial passages, at least 100 serial passages, or at least 150 serial passages. During the culturing, an increase in mutation frequency in the cultured cells is monitored over time, e.g., using random mutation capture assay. The candidate mutagenic compound (e.g., candidate mutagenic nucleoside analog) that causes an accumulation of mutations to exceed the threshold for viability can be considered mutagenic. Stated in another way, a mutagenic compound (e.g., mutagenic nucleoside analog) effective for lethal mutagenesis can increase the mutation frequency of a mammalian cell by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, at least about 100-fold, at least about 1000-fold or more, as compared to the mutation frequency of a mammalian cells in the absence of the mutagenic compound. In some embodiments, an effective mutagenic compound (e.g., mutagenic nucleoside analog) for lethal mutagenesis can increase the mutation frequency of a mammalian cell by at least about 2-fold when administered at, its half maximal inhibitory concentration for cell viability (IC50) or lower.

In some embodiments, the cells can be cultured in the presence of a mutagenic compound described herein (e.g., mutagenic nucleoside analog), in combination with at least one active agent, e.g., a therapeutic. In one embodiment, the active agent is another mutagenic compound e.g., a mutagenic nucleoside. For example, nucleoside analogs that are involved in different repair pathways can be selected, e.g., for a synergistic effect. When more than one mutagenic compound is used, the concentration of each mutagenic compound can be reduced, e.g., by about 20%, about 30%, about 40%, about 50%, about 60%, about 70% or more, as compared to the concentration of each nucleoside analog when used alone.

In some embodiments, a mutagenic compound described herein (e.g., mutagenic nucleoside analog) that induces lethal mutagenesis in cancer cell lines from one tissue can be effective in cancer cell lines from other tissues. However, it is possible that the time and/or the concentration of the mutagenic compound to achieve comparable cell death in cultures can vary with each cell type.

After in vitro screening, the identified mutagenic compounds (e.g., mutagenic nucleoside analogs) that increases the mutation rate of a mammalian cell, e.g., by at least about 2-fold, can be further assessed in vivo, e.g., in an appropriate animal model for a disease or disorder, e.g., cancer, to determine appropriate dosages, dosage frequency and therapeutic efficacy. For example, human cancer cell lines or small portions (e.g., about 0.5 mm³) of primary cancers or metastases obtained from a patient can be subcutaneously implanted into mice, e.g., in the highly vascular inguinal area of the hind legs of mice. The mice can be administered with the selected mutagenic compounds at different concentrations and/or on different administration schedules when the tumor size is doubled or larger. The mutagenic compound as described herein (e.g., mutagenic nucleoside analog) can be administered in capsules or added to drinking water. In some embodiments, the mutagenic compound (e.g., mutagenic nucleoside analog) can be administered in a sustained release, e.g., using an implantable osmotic pump or drug-loaded microparticles. In some embodiments, the mutagenic compound (e.g., mutagenic nucleoside analog) can be applied locally to the tumor, e.g., by injection. Tumor growth (e.g., tumor weight and size) can be monitored over the course of treatment. Any administered mutagenic compound that results in a decrease in average tumor volume can be considered effective, e.g., a decrease of at least about 20%, at least about 30%, at least about 40%, at least about 50%, at least about 60%, at least about 70%, at least about 80% or more, as compared to the tumor volume in the absence of the administration. In some embodiments, an effective mutagenic compound (e.g., mutagenic nucleoside analog) can decrease the tumor volume by at least about 90%, at least about 95%, at least about 97% or more, as compared to the volume of tumor in the absence of the administration. In one embodiment, an effective mutagenic compound (e.g., mutagenic nucleoside analog) can completely abolish the tumor. Biopsy or histology can be performed, for example, to confirm the in vivo results.

In various embodiments, mutagenic compounds as described herein do not inhibit replication or extension of a nucleic acid by a mammalian DNA polymerase. In such embodiments, the degree of a mutagenic compound inhibiting replication or extension of a nucleic acid by a mammalian DNA polymerase is less than 50%, and includes, for example, less than 40%, less than 30%, less than 20%, less than 10%, less than 5% inhibition or lower. In one embodiment, a mutagenic compound has a negligible inhibitory effect on replication or extension of a nucleic acid by a mammalian DNA polymerase. The term “replication,” as used herein, refers to a process in which a complementary strand of a nucleic acid sequence template is synthesized by a polymerase.

Any mutagenic compounds described herein (e.g., mutagenic nucleoside analogs) identified for lethal mutagenesis can be further modified to generate prodrugs thereof. Prodrugs are derivatives of the parent active drugs which have chemically or metabolically cleavable groups and become, under physiological conditions, pharmaceutically active in vivo.

The preparation of a prodrug involves a process of converting an active drug into inactive form. Such processes are well known to one skilled in the art. Prodrugs can be prepared, for example, by formation of ester, hemiesters, nitrate esters, amides, carbonate esters, carbamates to the active drug or by functionalizing the drug with azo, glycoside, peptide, and ether functional groups or use of polymers etc., as known to one skilled in the art.

Prodrugs can be prepared to alter the drug pharmacokinetics, improve the drug bioavailability by increasing absorption and distribution, decrease toxicity, increase duration of the pharmacological effect of the drug, or a combination thereof. In designing the prodrugs, one can consider factors such as the linkage between the carrier and the drug is usually a covalent bond, the prodrug is inactive or less active than the active parent, the prodrug is a reversible or bioreversible derivative of the drug, and the carrier moiety is non-toxic and inactive when released.

### Pharmaceutical Compositions and Routes of Administration

For in vivo administration, pharmaceutical compositions used for treatment of cancer are also described herein. The pharmaceutical composition includes at least one mutagenic compound described herein, and a pharmaceutically acceptable carrier. In one embodiment, the pharmaceutical composition includes at least one mutagenic nucleoside described herein, and a pharmaceutically acceptable carrier. In some embodiments, the pharmaceutical composition can further comprise at least one additional active agent as described earlier, e.g., an anti-neoplastic agent.

The term “pharmaceutically acceptable,” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” refers to a pharmaceutically-acceptable material, composition or vehicle for administration of an active agent, e.g., a mutagenic compound. Pharmaceutically acceptable carriers include any and all solvents, dispersion media, coatings, antibacterial and antifungal agents, isotonic and absorption delaying agents, and the like which are compatible with the activity of the active agent and are physiologically acceptable to the subject. Some examples of materials which can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alcohols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Pharmaceutically acceptable carriers can vary in a composition described herein, depending on the administration route and formulation. The mutagenic compounds and compositions described herein can be delivered via any administration mode known to a skilled practitioner. For example, the pharmaceutically acceptable composition described herein can be delivered in a systemic manner, via administration routes such as, but not limited to, oral, and parenteral including intravenous, intramuscular, intraperitoneal, intradermal, subcutaneous and intratumoral. In some embodiments, the compositions can be administered locally. In some embodiments, the compositions can be targeted to a tumor. For example, tumors such as breast or prostate tumors can be targeted by direct needle injection to the site of the lesion. Lung tumors can be targeted by the use of inhalation as a route of administration. Inhalation can be used in either systemic or local delivery. In one embodiment, the composition is targeted to a tumor, by direct intra-tumoral injection, e.g., injection into the tumor vasculature or local or regional administration relative to the tumor site. In one embodiment, the pharmaceutical composition is in a form that is suitable for injection. In another embodiment, the pharmaceutical composition is formulated for delivery by a catheter. In one embodiment, the pharmaceutical composition is formulated for oral administration.

When administering parenterally a pharmaceutical composition described herein, it can be generally formulated in a unit dosage injectable form (solution, suspension, emulsion). The pharmaceutical formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), suitable mixtures thereof. In some embodiments, the pharmaceutical carrier can be a buffered solution (e.g. PBS).

An oral composition can be prepared in any orally acceptable dosage form including, but not limited to, tablets, capsules, emulsions and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. Liquid preparations for oral administration can also be prepared in the form of a dry powder to be reconstituted with a suitable solvent prior to use.

In some embodiments, the pharmaceutical composition can be formulated in an emulsion or a gel. The gel pharmaceutical composition can be implanted locally to a region of a tumor.

In other embodiments, the mutagenic compounds or compositions can be administered in the form of sustained-release or controlled-release formulations, e.g., to reduce repeated administration and inconvenience to the patient. Many types of delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations thereof. Microcapsules of the foregoing polymers containing drugs are described in, for example, U.S. Pat. No. 5,075,109. Other examples include nonpolymer systems that are lipid-based including sterols such as cholesterol, cholesterol esters, and fatty acids or neuka1 fats such as mono-, di- and triglycerides; hydrogel release systems; liposome-based systems; phospholipid based-systems; silastic systems; peptide based systems; or partially fused implants. Specific examples include, but are not limited to, erosional systems in which the composition is contained in a form within a matrix (for example, as described in U.S. Pat. Nos. 4,452,775, 4,675,189, 5,736,152, 4,667,014, 4,748,034 and -29 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the composition. In addition, a pump-based hardware delivery system can be used to deliver one or more embodiments of the compositions described herein. Use of a long-term sustained release formulations or implants can be particularly suitable for treatment of chronic conditions, such as the suspected presence of dormant metastases. Long-term release, as used herein, means that a formulation or an implant is made and arranged to deliver a mutagenic compound described herein at a therapeutic level for at least 30 days, or at least 60 days. In some embodiments, the long-term release refers to a formulation or an implant being configured to deliver a mutagenic compound at a therapeutic level over several months.

In some embodiments, targeted delivery of the compositions described herein is desirable to reduce the potential side effects of the compositions on non-malignant or non-cancerous cells, e.g., normal healthy cells or benign tumor cells. In such embodiments, the addition of target moiety to the surface of delivery vehicles such as microspheres or microcapsules can specifically bring the mutagenic compound as described herein to the target cell. The particular cell surface targets that are chosen for the target moiety will depend upon the target cell. Cells can be specifically targeted, for example, by the use of antibodies against unique proteins, lipids or carbohydrates that are present on the cell surface. A skilled artisan can readily determine such molecules based on the general knowledge in the art. For example, certain tumors frequently possess a large amount of a particular cell surface receptor (e.g. neu with breast cancers), or an abnormal form of a particular protein. Therefore, a tumor antigen can serve as a specific target for delivering the compositions or formulations described herein into the tumor cells, to inhibit growth and/or proliferation of the tumor cells or to destroy the tumor cells. Any known tumor antigen expressed on the tumor cell surface can be used for generating an antibody to serve as a target moiety.

Examples of tumor antigens that can be used for generation of a targeting moiety include, but are not limited to, mini-MUC; MUC-1 (Marshall et al., J. CLin. Oncol. 18:3964-73 (2000); HER2/neu; HER2 receptor (U.S. Pat. No. 5,772,997); mammoglobulin (U.S. Pat. No. 5,922,836); labyrinthin (U.S. Pat. No. 6,166,176); SCP-1 (U.S. Pat. No. 6,140,050); NY-ESO-1 (U.S. Pat. No. 6,140,050); SSX-2 (U.S. Pat. No. 6,140,050); N-terminal blocked soluble cytokeratin (U.S. Pat. No. 4,775,620); 43 kD human cancer antigen (U.S. Pat. No. 6,077,950); human tumor associated antigen (PRAT) (U.S. Pat. No. 6,020,478); human tumor associated antigen (TUAN) (U.S. Pat. No. 5,922,566); L6 antigen (U.S. Pat. No. 5,597,707); carcinoembryonic antigen (RT-PCR analysis for breast cancer prognosis in Clin Cancer Res 6:4176-85, 2000); CA15-3 (Eur J Gynaecol Oncol 21:278-81, 2000); oncoprotein 18/stathmin (Op18) (Br J. Cancer 83:311-8, 2000); human glandular kallikrein (hK2) (Breast Cancer Res Treat 59:263-70, 2000); NY-BR antigens (Cancer Immun. March 30; 1:4, 2001), tumor protein D52 (Cancer Immun. March 30; 1:4, 2001), and prostate-specific antigen (Breast Cancer Res Treat 59:263-70, 2000); CD44, CD133, ABC7, c-kit, or SCA1, and EEA.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like.

The compositions can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation. With respect to compositions described herein, however, any vehicle, diluent, or additive used should have to be biocompatible with the active agents, e.g., mutagenic nucleoside analogs or other therapeutic agents.

The pharmaceutical compositions can be isotonic, i.e., they can have the same osmotic pressure as blood and lacrimal fluid. The desired isotonicity of the compositions described herein can be accomplished using sodium chloride, or other pharmaceutically acceptable agents such as dextrose, boric acid, sodium tartrate, propylene glycol or other inorganic or organic solutes. In one embodiment, sodium chloride is used in buffers containing sodium ions.

Viscosity of the compositions can be maintained at the selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

Typically, any additives (in addition to the mutagenic compounds described herein and/or additional active agents) can be present in an amount of 0.001 to 50 wt % solution in phosphate buffered saline, and the active ingredient is present in the order of micrograms to milligrams to grams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. For any therapeutic composition to be administered to a subject in need thereof, and for any particular method of administration, it is preferred to determine toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan.

Those skilled in the art will recognize that the components of the compositions should be selected to be biocompatible with respect to the active agent, e.g., mutagenic nucleoside analog. This will present no problem to those skilled in chemical and pharmaceutical principles, or problems can be readily avoided by reference to standard texts or by simple experiments (not involving undue experimentation).

In some embodiments, the compositions described herein can be prepared by mixing the ingredients following generally-accepted procedures. For example, at least one mutagenic nucleoside analog and optionally a therapeutic agent can be re-suspended in an appropriate pharmaceutically acceptable carrier and the mixture can be adjusted to the final concentration and viscosity by the addition of water or thickening agent and possibly a buffer to control pH or an additional solute to control tonicity. Generally the pH can vary from about 3 to about 7.5. In some embodiments, the pH of the composition can be about 6.5 to about 7.5. Compositions can be administered in dosages and by techniques well known to those skilled in the medical and veterinary arts taking into consideration such factors as the age, sex, weight, and condition of the particular patient, and the composition form used for administration (e.g., liquid). Dosages for humans or other mammals can be determined without undue experimentation by a skilled artisan.

### Selection of Subjects for Method of Treatment

In yet another aspect, provided herein are methods for treatment of any disease or disorder characterized by expression of a mutator phenotype. In some embodiments, the disease is cancer. Examples of cancers which can be treated by the methods and compositions as described herein include, but are not limited to, bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS associated leukemias and adult T-cell leukemia lymphoma; intraepithelial neoplasms including Bowen's disease and Paget's disease, liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilm's tumor. In one embodiment, the formulations comprising at least one mutagenic compound are administered for treatment of colon cancer. In another embodiment, the formulations comprising at least one mutagenic compound are administered for treatment of small cell lung cancer. In another embodiment, the formulations comprising at least one mutagenic compound are administered for treatment of prostate cancer. In another embodiment, the formulations comprising at least one mutagenic compound are administered for treatment of breast cancer. In another embodiment, the formulations comprising at least one mutagenic compound are administered for treatment of pancreatic cancer. In another embodiment, the formulations comprising at least one mutagenic compound are administered for treatment of liver cancer.

The terms “treatment” and “treating” as used herein, with respect to treatment of a disease, means preventing the progression of the disease, or altering the course of the disorder (for example, but are not limited to, slowing the progression of the disorder), or reversing a symptom of the disorder or reducing one or more symptoms and/or one or more biochemical markers in a subject, preventing one or more symptoms from worsening or progressing, promoting recovery or improving prognosis. For example, in the case of treating a cancer, therapeutic treatment refers to, e.g., a delay or inhibition of tumor growth, after administration of the compositions described herein. In another embodiment, the therapeutic treatment refers to alleviation of at least one symptom associated with cancer. Measurable lessening includes any statistically significant decline in a measurable marker or symptom, such as measuring the level of tumor marker, e.g., carcinoembryonic antigen (CEA), in a blood sample, or monitoring the tumor growth (as described in detail below) after treatment. In one embodiment, at least one symptom associated with cancer, is alleviated by at least about 10%, at least about 15%, at least about 20%, at least about 30%, at least about 40%, at least about 50% or greater. In another embodiment, at least one symptom is alleviated by more than 50%, e.g., at least about 60%, at least about 70% or greater. In one embodiment, at least one symptom is alleviated by at least about 80%, at least about 90% or greater, as compared to a control (e.g. in the absence of the composition described herein).

In one embodiment, subjects in need thereof are selected prior to administering the mutagenic compounds or the compositions described herein or employing the methods described herein. In some embodiments, the subject in need thereof can be diagnosed with a disease or disorder associated with a mutator phenotype as described herein. In some embodiments, the subject in need thereof can be diagnosed with cancer as disclosed herein.

In some embodiments, the pharmaceutical compositions comprising at least one mutagenic compound described herein can be administered to a subject who has cancer and has been exposed to or treated with other cancer therapies. In those embodiments, the pharmaceutical compositions comprising at least one mutagenic compound can be administered to a subject alone or in combination with other cancer therapies such as surgery, chemotherapy, radiotherapy (both brachytherapy and teletherapy), thermotherapy, immunotherapy, hormone therapy and laser therapy, to provide a beneficial effect, e.g. reducing tumor size, slowing rate of tumor growth, reducing cell proliferation of the tumor or cancer cells, promoting cancer cell death, inhibiting metastasis, inhibiting angiogenesis at the tumor or cancer site, or otherwise improving overall clinical condition.

Due to the potential consequences associated with the use of lethal mutagenesis, particularly risk of secondary tumors, the methods and compositions described herein can, in one embodiment, be applicable to specific circumstances. For example, in some embodiments, the pharmaceutical compositions comprising at least one mutagenic compound described herein can be administered to a subject who has a therapy-resistant cancer, for example a chemotherapy resistant cancer, alone or in combination with other anti-cancer agents or therapies. In some embodiments, the methods and compositions herein can be applicable to a subject diagnosed with cancer that is or has become resistant to or non-responsive to at least one or more anti-cancer therapies indicated for that type of cancer. In certain embodiments, the methods and compositions herein can be applicable to a subject diagnosed with cancer that is or has become resistant to or non-responsive to at least two or more anti-cancer therapies indicated for that type of cancer. In certain embodiments, the methods and compositions herein can be applicable to a subject diagnosed with cancer that is or has become resistant to or non-responsive to at least three or more anti-cancer therapies indicated for that type of cancer. In certain embodiments, the methods and compositions herein can be applicable to a subject diagnosed with cancer that is or has become resistant to or non-responsive to all available cancer therapies indicated for that type of cancer. In some embodiments, elderly individuals in which the induction of new tumors is less likely to be consequential are also amenable to the methods and compositions described herein.

In some embodiments, the pharmaceutical compositions comprising at least one mutagenic compound described herein can be administered to a subject for treatment of cancer that is highly likely to recur in a period of time, e.g., less than a month, less than two months, less than three months, less than four months, less than five months, less than six months, less than seven months, less than eight months, less than nine months, less than ten months, less than eleven months, less than one year, less than two years, less than three years, less than four years, or less than five years. The phrase “highly likely to recur” in reference to a cancer, as used herein, means that the probability of cancer recurrence in a subject within a period of time is at least about 50%, at least about 60%, at least about 70%, at least about 80%, at least about 90%, at least about 95%, at least about 98% or higher.

In some embodiments, the pharmaceutical compositions comprising at least one mutagenic compound described herein can be administered to a subject for treatment of the cancer type that is known to be highly non-responsive to at least one or more anti-cancer therapies, e.g., including at least two or more anti-cancer therapies, at least three or more anti-cancer therapies, at least four or more anti-cancer therapies, at least five or more anti-cancer therapies, or even all available anti-cancer therapies in a standard therapeutic regimen, e.g., all available FDA approved anti-cancer therapies for that particular cancer. The phrase “highly non-responsive to anti-cancer agents,” in reference to cancer, is used herein to describe that the likelihood of a cancer to be treated with anti-cancer agents is less than 50%, less than 40%, less than 30%, less than 20%, less than 10%, less than 5% or lower. The phrase “highly non-responsive to anti-cancer agents” can also describe cancers that recur or develop resistance against at least one anti-cancer agent. In some embodiments, the phrase “highly non-responsive to anti-cancer agents,” can mean that a significant portion of cancer cells within a tumor, e.g., more than 10%, more than 20%, more than 30%, more than 40%, more than 50%, more than 60%, more than 70%, more than 80%, more than 90%, more than 95% or higher, are not killed or continue to proliferate. The determination of whether a cancer is “non-responsive” can be determined either in vivo or in vitro by any method known in the art for assaying the effectiveness of treatment on cancer, e.g., monitoring the cancer or tumor growth or level of tumor biomarkers in blood. In one embodiment, a cancer is “highly non-responsive to anti-cancer agents” when the size of tumor has not been significantly reduced, e.g., by about 10%, about 20%, about 30%, about 40%, about 50%, about 60%, about 70%, about 80%, about 90%, about 95% or higher, as compared to the size of the tumor in the absence of the treatment, or has indeed increased during the treatment. Exemplary types of cancer known to be highly non-responsive to anti-cancer therapies, such as chemotherapies, include, but are not limited to, pancreatic cancer, brain cancer, melanoma of the skin, bladder cancer, kidney cancer, anaplastic thyroid carcinoma, lung cancer or any Stage IV cancer, such as Stage IV prostate cancer, which has generally metastasized, or spread to other organs or throughout the body. In one embodiment, the mutagenic compounds and compositions described herein can be administered to a subject who is diagnosed with or suffering from pancreatic cancer.

In some embodiments, subjects whose cancer has repeatedly recurred after treatments with multiple anti-cancer therapies, e.g., anti-cancer chemotherapeutic agents, for example, at least two, at least three, at least four, at least five or more anti-cancer therapies, can be subjected to a mutagenic compound or a pharmaceutical composition comprising a mutagenic compound as described herein. By “repeatedly recurred” or “repeated recurrence” is meant that repeated cycles of cancer treatment and cancer recurrence have occurred in the subject, i.e., a tumor after initial treatment with anti-cancer therapies and after a period of time during which the cancer cannot be detected has returned and been treated with anti-cancer therapies again, but the cancer still recurs thereafter.

In some embodiments, subjects diagnosed with cancer that is only responsive to highly toxic anti-cancer therapies can be subjected to or administered a mutagenic compound or a pharmaceutical composition comprising such mutagenic compound described herein. As used herein, in reference to a cancer or a tumor, the phrase “only responsive to highly toxic anti-cancer therapies” indicates that the cancer or tumor does not respond, or responds poorly, to standard dosages of anti-cancer therapies, such as anti-cancer chemotherapeutic agents, and requires higher dosages and/or frequencies of administration than is typical for that cancer, thus resulting in increased toxicity, i.e., side-effects, in the subject. Such increased toxicity is a result of the anti-cancer therapy having toxic effects on non-cancerous cells, such as liver cells or heart cells, in addition to the cancer cells, such that sufficient damage or cell death is caused by the therapy to non-cancerous cells in the subject, thus mitigating any effects on the tumor or cancer. A “highly toxic anti-cancer therapy” for a given cancer is one that results in serious side-effects or toxicities, such as myelosuppression, which can result in potentially fatal, systemic infections; platelet deficiencies; cardiotoxicity; hepatotoxicity; nephrotoxicity; gonadotoxcity; or encephalopathy, that requires the temporary or permanent cessation of that therapy in the subject, i.e., the toxic and/or fatal risks caused by continued administration of the anti-cancer therapy is greater than the risks caused by continued cancer growth or further cancer progression in the subject.

In some embodiments, the number of preexisting mutations in individual tumors can be determined to assess the utility of mutagenic compounds described herein, e.g., mutagenic nucleosides/nucleotides, as therapy. For example, a biopsy of a tumor followed by genome sequencing can be performed to determine preexisting mutations in a tumor. If a subject is diagnosed with a tumor having a large number of mutations e.g., more than 1000 somatic mutations per genome, more than 5000 somatic mutations per genome, more than 10,000 somatic mutations per genome, or more than 50,000 somatic mutations per genome, the subject can be administered with a mutagenic compound or a pharmaceutical composition comprising such compound.

In some embodiments, a mutagenic compound described herein can be administered to a subject when the subject is diagnosed with a tumor having an increase in mutation frequency of tumor cells of at least about 2-fold, at least about 5-fold, at least about 10-fold, at least about 25-fold, at least about 50-fold, at least about 100-fold, at least about 150-fold, or at least about 200-fold, as compared to the mutation frequency of normal cells, e.g., from the normal tissue of the same subject, or from a normal healthy person.

In one embodiment, the methods and compositions herein can be administered to subjects who are diagnosed with or at risk of having malignant cancer or metastasis, or in whom a cancer has metastasized to multiple sites. As used herein, the term “metastases” or “metastatic tumor” refers to a secondary tumor that grows separately elsewhere in the body from the primary tumor and has arisen from detached, transported cells, wherein the primary tumor is a solid tumor. The primary tumor, as used herein, refers to a tumor that originated in the location or organ in which it is present and did not metastasize to that location from another location. As used herein, the term “malignant cancer” is one having the properties of invasion and metastasis and showing a high degree of anaplasia. Anaplasia is the reversion of cells to an immature or a less differentiated form, and it occurs in most malignant tumors. Methods for diagnosis of metastasis are well known to a skilled practitioner. Such methods include, but are not limited to, blood tests for detection of tumor markers for specific cancer (e.g., Carcinoembryonic Antigen (CEA); Alpha-fetoprotein (AFP); CA-125; CA19-9; CA15-3; prostate-specific antigen (PSA); human chorionic gonadotropin (HCG), hormones (e.g., ACTH or ADH); acid phosphatase; neuron specific enolase; EphA2; and galactosyl transferase II); CELLSEARCH® Circulating Tumor Cell Test (from BioVantra); X-rays and other imaging studies such as bone scan, MRI scan, CAT scan and CT scans. In some embodiments, genetic tests can be performed for diagnosis of metastatic tumors, e.g., by detecting the presence or absence of genes or single nucleotide polymorphisms (SNPs) associated with cancer that are recognized in the art. In some embodiments, a subject to be treated with the compositions and methods described herein can have one or more, i.e., multiple, sites of metastasis, one or more of which sites are not responsive to anti-cancer therapies, as the terms are defined herein.

In yet other embodiments, the mutagenic nucleoside analog in a suitable formulation can be administered to subjects who exhibit symptoms of cancer (e.g., early or advanced).

In some embodiments, subjects diagnosed with cancer having a highly microsatellite instable (MSI-H) phenotype are amenable to the methods of treatment described herein.

As used herein, a “subject” can mean a human or an animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient”, “individual”, and “subject” are used interchangeably herein. A subject can be male or female.

In one embodiment, the subject is a mammal. The mammal can be a human, non-human primate, mouse, rat, dog, cat, horse, or cow, but are not limited to these examples. Mammals other than humans can be advantageously used as subjects that represent animal models of cancer therapy. In addition, the methods and compositions described herein can be employed in domesticated animals and/or pets.

### Some Selected Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the aspects described herein, in connection with percentages means±1%.

In one respect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

The term “lethal mutagenesis,” as used herein, refers to a process in which the number of mutations induced in a cell exceeds an error threshold, beyond which the cell is no longer viable. As described herein, lethal mutagenesis is induced in cancer cells as a therapeutic treatment of cancer.

The term “error threshold,” as used herein refers to a critical mutation frequency, above which cancer cells are no longer viable.

The term “error catastrophe,” as used herein, refers to the extinction of a population as a result of excessive genomic mutations. In some embodiments, when referring to cancer, the error catastrophe refers to a phenomenon, in which the mutations in a cancer cell reach above the error threshold and thus natural selection (i.e., Darwinian selection) ceases to operate.

The term “quasispecies,” as used herein, when referring to cancer, means a population of cancer cell variants that have accumulated large numbers of random and independent mutations and that cooperate to contribute to maintain the population. For example, in a quasispecies, vigorously replicating variants assure the survival of the entire population, while the poorly replicating variants provide genetic flexibility to facilitate survival within, and rapid adaptation to, a changing selective environment.

The term “resistance mutation,” as used herein, refers to mutations in DNA of a cell induced by a selection process, e.g., with a drug. These mutations are generally present at low frequency prior to selection. For example, after cancer cells have been treated with a chemotherapeutic drug for a period of time, cancer cells with resistant mutations that enable the cells survive in the presence of the drug are selected.

The term “nucleic acid molecule” is well known in the art. A “nucleic acid molecule” as used herein generally refers to a molecule (i.e., strand) of DNA, RNA or a derivative or analog thereof, comprising a nucleobase. A nucleobase includes, for example, a naturally occurring purine or pyrimidine base found in DNA (e.g. an adenine “A,” a guanine “G” a thymine “T” or a cytosine “C”) or RNA (e.g. an A, a G. an uracil “U” or a C). The term “nucleic acid molecule” encompasses the terms “oligonucleotide” and “polynucleotide,” each as a subgenus of the term “nucleic acid molecule.” The term “oligonucleotide” refers to a molecule of between about 3 and about 100 nucleobases in length. The term “polynucleotide” refers to at least one molecule of greater than about 100 nucleobases in length.

As used herein, the term “mutagenic compound” refers to a compound that can increase the mutation frequency in the genome of a mammalian cell, influence the fidelity of a nucleic acid replication process, influence a nucleic acid repair process, and/or influence the balance of the nucleotide pool.

The terms “peptide” and “protein” are used interchangeably herein to refer to a polymer or oligomer of consecutive amino acid residues. As used herein, the term “peptide’ refers to a short polymer formed from the linking of amino acids. A “peptide” is at least 4, or at least 5 amino acids and no more than to 50 amino acids in length.

The term “complementary” as used herein refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand which is anti-parallel to the first strand if the residue is guanine. Accordingly, the term “non-complementary,” as used herein, means that an adenine residue of a first nucleic acid region does not form a specific hydrogen bond with a thymine or uracil residue (“base mispair”) of a second nucleic acid region which is anti-parallel to the first region. Similarly, a base mispair forms when a cytosine residue of a first nucleic acid strand does not base-pair with a guanine residue of a second nucleic acid strand which is anti-parallel to the first strand.

The term “polymerase,” as used herein refers to an enzyme (DNA or RNA polymerase) that synthesizes a polynucleotide sequence complementary to a pre-existing template polynucleotide (DNA or RNA). In one embodiment, the polymerase is a DNA polymerase. DNA polymerases are enzymes that are capable of incorporating nucleotides into the 3′ hydroxyl terminus of a nucleic acid in a 5′ to 3′ direction thereby synthesizing a nucleic acid sequence.

As used herein, the term “tumor” refers to a mass of transformed cells that have escaped the host's mechanisms for the control of cell position and proliferation. The transformed cells are characterized by neoplastic uncontrolled cell multiplication which is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to invade and/or “metastasize” (i.e., a metastatic tumor), a tumor also can be nonmalignant (i.e. non-metastatic tumor). Tumors are hallmarks of cancer, a neoplastic disease the natural course of which is frequently fatal. Cancer cells exhibit the properties of invasion and metastasis and are highly anaplastic.

The term “antibody” is meant to be an immunoglobulin protein that is capable of binding an antigen. Antibody as used herein is meant to include antibody fragments, e.g. F(ab′)2, Fab′, Fab, capable of binding the antigen or antigenic fragment of interest.

As used herein, the term “administer” or “administration” refers to the placement of a composition into a subject by a method or route which results in at least partial localization of the composition at a desired site such that desired effect is produced. Routes of administration suitable for the methods described herein include, but are not limited to, orally or by injection. Generally, local administration results in more of the composition being delivered to a specific location as compared to the entire body of the subject.

The term “therapy-resistant” as used herein, refers to a cancer present in a subject that is resistant to, refractory to, or non-responsive to a therapy or combination of therapies, and means that the subject has been treated with at least one or more, at least two or more, at least three or more, or even all anti-cancer therapies available in a standard therapeutic regimen for a particular cancer that did not provide effective treatment as the term is defined herein, or that did not provide lasting effective treatment. In one embodiment, a cancer diagnosed as therapy resistant is non-responsive to at least one, at least two, at least three, or even all available (e.g., all US FDA approved therapies for that particular cancer) anti-cancer therapies. In such embodiment, a cancer that is non-responsive to anti-cancer therapy or therapies is manifested, e.g., by continued growth of the tumor or proliferation of cancer cells at the same rate or at a higher rate, and/or continued progression of the tumor, such as formation of one or more metastases.

The terms “anti-cancer agent” or “anti-cancer therapy,” as used interchangeably herein, refer to an agent or a therapy useful in treating cancer, e.g., an agent or a therapy capable of negatively affecting a tumor and/or cancer cells in a subject, for example killing cancer cells, inducing apoptosis in cancer cells, reducing the growth rate of cancer cells, reducing the number of metastatic cells, reducing tumor size, inhibiting tumor growth, reducing blood supply to a tumor or cancer cells, promoting an immune response against cancer cells or a tumor, preventing or inhibiting the progression of cancer, or increasing the lifespan of the subject with cancer. Examples of anti-cancer therapeutic agents include, but are not limited to, e.g., surgery, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the methods described herein.

The term “cytotoxic agent” as used herein refers to a substance that inhibits or prevents the function of cells and/or causes destruction of cells. The term is intended to include radioactive isotopes (e.g. At211, I131, I125, Y90, Rel186, Rel88, Sm153, Bi212, P32 and radioactive isotopes of Lu), chemotherapeutic agents, and toxins such as small molecule toxins or enzymatically active toxins of bacterial, fungal, plant or animal origin, including fragments and/or variants thereof.

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous anti-neoplastic drugs. Most if not all of these agents are directly toxic to cancer cells and do not require immune stimulation. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2.sup.nd ed., .COPYRGT. 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

The term “recur” or “recurrence,” when referring to cancer, is defined herein as a return of cancer after treatment with anti-cancer therapies and after a period of time during which the cancer cannot be detected. The period of time as defined herein can be less than a month, less than two months, less than three months, less than four months, less than five months, less than six months, less than seven months, less than eight months, less than nine months, less than ten months, less than eleven months, less than one year, less than two years, less than three years or longer. The same cancer can return in the same location where it first started or in another location of the body. For example, prostate cancer can return in the area of the prostate gland, or it can recur in the bones. Either situation is considered as a cancer recurrence.

As used herein, the term “pro-drug” refers to any compound which releases an active parent drug in vivo when such pro-drug is administered to a mammalian subject. Pro-drugs of a compound are typically prepared by modifying one or more functional group(s) present in the compound in such a way that the modification(s) can be cleaved in vivo to release the parent compound. Examples of pro-drugs include, but are not limited to, esters (e.g., acetate, formate, and benzoate derivatives) and carbamates (e.g., N,N-dimethylaminocarbonyl) of hydroxy functional groups, and amides, carbamates and urea derivatives of amino functional groups, and the like. Pro-drug forms often offer advantages of solubility, tissue compatibility, or delayed release in the mammalian organism. See Bundgard, Design of Prodrugs, 7-9, 21-24 (Elsevier, Amsterdam, 1985); Silverman, Organic Chem. of Drug Design & Drug Action, 352-401 (Academic Press, San Diego, Calif.). Moreover, the prodrug derivatives can be combined with other features known to one skilled in the art to enhance bioavailability.

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate plane polarized light in different directions). Enantiomers generally have identical physical properties, such as melting points and boiling points, and also have identical spectroscopic properties. Enantiomers can differ from each other with respect to their interaction with plane polarized light and with respect to biological activity.

The term “enantiospecific” as used herein refers to the absolute configuration of the molecule about its chiral center(s) denoted by “L” and “D”. The designations may appear as a prefix or as a suffix; they may or may not be separated from the isomer by a hyphen; they may or may not be hyphenated; and they may or may not be surrounded by parentheses.

The term “racemic mixture” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is highly preferred that a small molecule have a molecular mass equal to or less than 700 Daltons.

The structure definitions such as “alkyl” are provided below for nomenclature purposes. They do not exclude the meaning as those acquired in the art to which the aspects described herein pertain. The term “alkyl” as used herein refers to a linear or branched saturated hydrocarbon group typically containing 1 to about 4 carbon atoms, such as methyl, ethyl, n-propyl, isopropyl, n-butyl, isobutyl, sec-butyl, and tert-butyl.

As used herein, the term “heteroalkyl” refers to alkyl in which at least one carbon atom is replaced with a heteroatom. The term “haloalkyl” as used herein refers to an alkyl structure with at least one substituent of fluorine, chorine, bromine or iodine, or with combinations thereof. If not otherwise indicated, the term “alkyl” includes linear, branched, cyclic, unsubstituted, substituted, and/or heteroatom-containing alkyl, respectively.

The term “alkenyl” as used herein refers to a linear, or branched hydrocarbon group of 2 to about 4 carbon atoms containing at least one double bond, such as ethenyl, n-propenyl, isopropenyl, n-butenyl, and isobutenyl.

The term “alkynyl” as used herein refers to a linear or branched-chain hydrocarbon group having one or more carbon-carbon triple-bonds and having from 2 to about 4 carbon atoms. Examples of alkynyl groups include ethynyl, propynyl, and butynyl.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 12 carbon atoms. Exemplary aryl groups contain one aromatic ring or two fused or linked aromatic rings, e.g., phenyl, naphthyl, biphenyl, diphenylether, diphenylamine, and benzophenone.

As used herein, the term “heteroaryl” refers to aryl substituents in which at least one carbon atom is replaced with a heteroatom such as oxygen, nitrogen and sulfur. The term “heteroaryl” includes ring systems such as pyridine, quinoline, furan, thiophene, pyrrole, imidazole and pyrazole.

The term “heterocycle” as used herein refers to a single ring or multiple rings that are fused together, directly linked, or indirectly inked (such that the different rings are bound to a common group such as a methylene or ethylene moiety), in which at least one carbon atom is replaced with a heteroatom such as oxygen, nitrogen and sulfur. Preferred heterocycyl groups contain 5 to 15 carbon atoms. For example, a heterocycle can be a five-membered ring with at least one carbon replaced by oxygen or nitrogen.

The term “cyclic” refers to aromatic groups that may or may not be substituted and/or heteroatom containing, and that may be monocyclic, bicyclic, or polycyclic.

By “substituted” as in “substituted alkyl,” “substituted aryl,” and the like, is meant that in the alkyl, aryl, or other moiety, at least one hydrogen atom bound to a carbon (or other) atom is replaced with one or more non-hydrogen substituents.

The term “halogen” as used herein refers to fluorine, chlorine, bromine, and iodine.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

Unless otherwise defined, all technical and scientific terms used herein have the same meaning as commonly understood by one of ordinary skill in the art to which the aspects described herein belongs. Although methods and materials similar or equivalent to those described herein can be used in the methods for identification of mutagenic compounds (e.g., mutagenic nucleoside analogs) that induce lethal mutagenesis in human tumors as featured herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Example 1

### Selection of Mutagenic Nucleoside Analogs

Many factors have to be considered for selecting of nucleosides for the induction of lethal mutagenesis; these include an analogs capacity for ambiguous base pairing, the extent by which the substituted group inhibits phosphorylation by cellular nucleoside/nucleotide kinases and incorporation of the analog by DNA polymerases, as well as stability, solubility, availability and cost. In principle, unnatural hydrophobic nucleosides that resemble canonical nucleotides in DNA (Lu et al., 8 Org Biomol Chem. 2704 (2010); Hwang et al., 37 Nuclei Acids Res 4757 (2009); Seo et al., 131 J Am Chem Soc 3246 (2009)) with ambiguous base pairing properties could be mutagenic; however, there is a lack of evidence indicating any of these is incorporated into DNA in vivo.

One candidate mutagenic compound for assessment of its mutagenic potential is O⁴-methyl thymidine. The choice of O⁴-mT is based on the following considerations: First, O⁴-mT triphosphate is incorporated by a variety of DNA polymerases (Preston et al., 83 PNAS 8501 (1986)). Second, the repair of O⁴-mT residues in DNA is inefficient in mammalian cells (Singer et al. 84 IARC Sci Publ. 37 (1987)); the t_{1/2} in mammalian cells is 20 hrs to 80 hrs (Brent et al., 85 PNAS 1759 (1988)). Third, copying past site specific O⁴-mT residues by DNA polymerases in vitro results in a 20% increase in mis-incorporation (Singer et al. 84 IARC Sci Publ. 37 (1987)). Fourth, mis-incorporation of opposite O⁴-mT in vivo results in AT->GC transitions. Finally, O⁴-mT has been previously shown to induce lethal mutagenesis of HIV in tissue culture (Loeb et al. 96 PNAS 1492 (1999)), but was not considered for use against HIV because of the evidence that it was also stably incorporated into the host DNA genome (Anderson et al., 58 Annu. Rev. Microbiol. 183 (2004)).

Other examples of candidate mutagenic compounds for evaluation of mutagenic potential include, but are not limited to, modified deoxyguanosines. Without limitations, modifications of guanines that are expected to base pair ambiguously with T or C residues in DNA include, O⁶-alkyl guanines (e.g., O⁶-methyl guanine, O⁶-ethyl guanine, O⁶-isopropyl guanine); replacement of N³ with another atom (e.g., C, O, and S), and additions of C⁸ by a halogen atom (e.g., F, Cl, Br), OH, SH or alkyl groups (e.g., CH₃), and any combination thereof (FIG. 2).

To evaluate the mutagenic potential of nucleoside analogs, the Random mutation Capture assay can be used. The PIG-A assay can present an alternative approach (Araten et al., 108 Blood 734 (2006)).

### Example 2

### Induction of Lethal Mutagenesis

In aspects described herein, growth of cancer cells in cultures containing mutagenic nucleoside analogs can result in the accumulation of nuclear mutations until a critical number is obtained resulting in an error catastrophe and ablation of the cell population.

A panel of nucleoside analogs is screened to identify effective mutagenic analogs, e.g., at least 10, at least 20, at least 30, at least 40, at least 50, at least 100, at least 150, at least 200 nucleoside analogs or more, can be screened using one or more, e.g., at least two, at least three, at least four, at least five or at least six different cell lines. The highest concentration of each analog at which there is no apparent toxicity is first established. Cells are monitored for analog incorporation and induced mutagenesis using the PIG-A assay. After 20 sequential transfers, the accumulation of mutations in nuclear and mitochondrial DNAs is measured using the random mutation capture assay (FIG. 3).

To determine nucleotide analog concentration, human colon cancer cells, e.g., mismatch repair-proficient cells such as SW480 and SW620 (McNutt et al., 44 Lab Invest 309 (1981)), and mismatch repair-deficient cells such as SW48 and HCT116 (Branch et al., 55 Cancer Res 2304 (1995); Casares et al. 11 Oncogene 2303 (1995)) have been cultured for 48 hr in the presence of increasing concentration of each of the analogs, and viability have been measured using crystal violet staining (Table 2). The concentration of each analog used in the serial transfer experiments can be 50% less than the highest dose at which there is no reduction in viability. Analogs that fail to be toxic can be used at 100 μM.

TABLE 2
Nucleoside toxicities for SW480 and HCT116 cell lines

| | |
|---|---|
| Highest dose | decreased |
| without | viability |
| SW480 | HCT116 |

| | | | | |
|---|---|---|---|---|
| KP-1212 | 3.3 | mM | 3.3 | mM |
| O⁴-methyl-2′-deoxythymidine | >3.3 | mM | >3.3 | mM |
| O⁴-ethyl-2′-deoxythymidine | >3.3 | mM | 330 | uM |
| 5-hydroxy-2′-deoxycytidine | 300 | uM | 30 | uM |
| 2′-deoxyinosine | >3.3 | mM | >3.3 | mM |
| 2′-deoxyaminopurine | >3.3 | mM | >3.3 | mM |
| 2′-aminopurine | 330 | nM | 330 | nM |
| ENU | 0.6 | mg/ml | 1.1 | mg/ml |

In Table 2, mismatch repair proficient and deficient cell lines were assayed for viability after growing for 48 hr with the indicated analogs in nM to mM concentrations.

To perform serial transfer experiments, serial passage of cell lines are carried out in parallel duplicate 96-well plates. Each well can be seeded with approximately 7000 cells in 150 μl of media (DMEM, 15% serum, penicillin, streptomycin and L-glutamine) and nucleoside analogs. Each analog is analyzed in triplicate using five different cell lines (vide infra). After two doublings (˜3 days) the media is removed and the cells are washed, detached with trypsin, counted and split 1:4 into media containing fresh nucleoside analog.

A sensitive method such as the random mutation capture assay (RMC) can be used to quantify mutation frequency. For example, a series of target enrichment steps can be employed to ensure complete digestion of non-mutant (wild type) sequences. Alternatively, by carefully limiting the amount of DNA to be digested by the restriction enzyme reaction, these enrichment steps can be omitted. This has the added advantage of allowing multiple target sequences to be interrogated simultaneously and reduces the overall amount of DNA required to perform the assay. With nuclear DNA from normal fibroblasts, one mutation in 10⁸ nucleotides can be detected (Bielas et al., 2 Nat Meth 285 (2005)); with mitochondrial DNA, the frequency of point mutations is 10⁻⁶ (Vermulst et al. 39 Nat Genet 540 (2007); Vermulst et al. 40 Nat Genet 392 (2008)). Using this assay, it has been previously demonstrated that exposure of cells to differential amounts of ethylnitrosourea (ENU) results in a linear increase in mutagenesis and the spectrum of induced mutations is similar to those reported in the literature (Bielas et al., 2 Nat Meth 285 (2005)). Furthermore, the random mutation frequency at the Taqα1 site in intron 6 of the p53 gene has been measured in matched pairs of cancer and normal tissue from five patients, in a coded protocol (Bielas et al. 103 PNAS 18238 (2006)). Mutations were detected in only one normal tissue sample. In contrast, the mutation frequency in the tumor tissues was elevated by about 65-fold to about 475-fold. The values obtained from normal human tissues and lung tumors are similar to those reported in Fen I-deficient cells (Zheng et al. 13 Nature Med 812 (2007)).

### Example 3

### Efficiency and Spectrum of Nucleoside Analogs

For nucleosides that induce lethal mutagenesis, it is determined if chemical modification of the analog structure can further enhance the induction of mutations. In order to select the most efficacious analog for further studies, different chemical modifications are introduced into a parent nucleoside. The corresponding nucleoside triphosphates are synthesized and the kinetics of incorporation of nucleoside triphosphate are directly measured. Rates of incorporation are determined using purified human DNA polymerase δ (Schmitt et al. 91 Biochimie 1163 (2009)). Mis-incorporation is determined using the M13 gap filling assay (Kunkel et al., 261 J Biol Chem 160 (1986); Kunkel, 279 J Bio Chem 16895 (2004)). If any of the modified nucleotides is incorporated more effectively or is more mutagenic than the parent mutagenic compound, the toxicity and serial transfer experiments described herein can be repeated, respectively.

A combination of identified nucleoside analogs that induce lethal mutagenesis can also be used to determine if cells in culture get killed more rapidly. In one embodiment, analogs that are repaired by different repair pathways can be employed. Such analogs can be more likely to exhibit additive (or possibly synergistic) effects. Mixtures of at least two nucleotide analogs are analyzed in serial transfer experiments.

Each of the analogs that ablates colon cancer cells in culture are tested in at least six established cell lines derived from other human tumors. Examples of those cells lines include, but are not limited to, small cell lung cancer (DMS-114); prostate cancer (MCI-7); breast cancer (MCF7); pancreatic cancer (Rossi); and liver cancer (HepG2). While the time to achieve ablation in cultures can vary with cell type, analogs that induce lethal mutagens are envisioned to be effective against a variety of cells from different tissues.

### Example 4

### Effect of Nucleoside Analogs on Tumor Growth In Vivo

The protocol enumerated by Morton and Houghton is followed for both cultured human cancer cells and human tumors (Morton et al., 2 Nat Protoc 247 (2007)). Experiments are carried out in athymic nude male mice of 25 days in age obtained from the Jackson Laboratories and maintained under barrier conditions at University of Washington.

Preliminary studies are carried out using human colon cancer cell lines and analog combinations in which cell death has been demonstrated in serial transfer experiments. It is sought to determine if the cell culture experiments can be mimicked using heterotransplants. Male nude mice are injected subcutaneously at multiple paired sites with 0.1 ml of culture medium with and without about 10⁷ cells. Tumor growth is quantified every other day. When any tumor doubles in volume, the nucleoside analog is given in capsules or added to the drinking water and the effects on tumor growth is determined. Each analog/cell line pair is assessed in triplicate at three analog concentrations.

Further, small portions of primary colon cancers or liver metastases are obtained at the time of surgery from patients who have undergone tumor reduction after chemotherapy. Informed consent is obtained and the patient's identity is safeguarded. Fragments of each tumor of approximately 0.5 mm³ are implanted subcutaneously in the highly vascular inguinal area of the two hind legs of each immunocompromised mouse, and after one of the tumors increases in size by more than 50%, the selected nucleoside analog is administered. Each tumor is implanted in ten mice and exposed to three different concentrations of each of the analogs to be studied. Mice are treated for up to four weeks or less depending on whether a significant difference in tumor volume is detected in analog-treated versus placebo-treated mice. After eight weeks of treatment, final tumor volumes are determined and all remaining mice are euthanized. Any analog that results in a 50% decrease in average tumor volume is considered effective and confirmed by biopsy and histology.

Without wishing to be bound by theory, cancers can be uniquely sensitive to mutagenic nucleosides because of pre-existing mutation burden. Presented herein is a comprehensive approach of identifying mutagenic nucleosides that can ablate tumor cells in culture and an in vivo animal model that mimics tumor growth in patients.

### REFERENCES I

1. Loeb L A, Springgate C F, Battula N. Errors in DNA replication as a basis of malignant changes. Cancer Res 1974; 34: 2311-21.
2. Loeb L A. Mutator phenotype may be required for multistage carcinogenesis. Cancer Res 1991; 51: 3075-9.
3. Loeb L A, Loeb K R, Anderson J P. Multiple mutations and cancer. Proc Natl Acad Sci USA 2003; 100: 776-81.
4. Beckman R A and Loeb L A. Negative clonal selection in tumor evolution. Genetics 2005; 171: 2123-31
5. Beckman R A and Loeb L A. Efficiency of carcinogeneis with and without a mutator mutation. Proc Natl Acad Sci USA 2006; 103: 15 140-5.
6. Beckman R A. Mutator mutations enhance tumorigenic efficiency across fitness landscapes. Plos One 2009; 4: e5860.
7. Boveri T. Uber mehrpolige Mitosen als Mittel zur Analyse des Zellkerns. Veh Dtsch Zool Ges Wurzburg 1902.
8. Mitelman F. Recurrent chromosome aberrations in cancer. Mutat Res 2000; 462: 247-53.
9. Kallioniemi A, Kallioniemi O P, Sudar D, Rutovitz D, Gray J W, Waldman F, et al. Comparative genomic hybridization for molecular cytogenetic analysis of solid tumors. Science 1992; 258: 818-21.
10. Jones S, Zhang X, Parsons D W, Lin J C, Leary R J, Angenendt P, et al. Core signaling pathways in human pancreatic cancers revealed by global genomic analyses. Science 2008; 321: 1801-6.
11. Kallioniemi A, Kallioniemi O P, Piper J, Tanner M, Stokke T, Chen L, et al. Detection and mapping of amplified DNA sequences in breast cancer by comparative genomic hybridization. Proc Natl Acad Sci USA 1994; 91: 2156-60.
12. Wood L D, Parsons D W, Jones S, Lin J, Sjoblom T, Leary R J, et al. The genomic landscapes of human breast and colorectal cancers. Science 2007; 318: 1108-13.
13. Klein C A, Schmidt-Kittler O, Schardt J A, Pantel K, Speicher M R, Riethmuller G. Comparative genomic hybridization, loss of heterozygosity, and DNA sequence analysis of single cells. Proc Natl Acad Sci USA 1999; 96: 4494-9.
14. Aaltonen L A, Peltomaki P, Leach F S, Sistonen P, Pylkkanen L, Mecklin J P, et al. Clues to the pathogenesis of familial colorectal cancer. Science 1993; 260: 812-6.
15. Thibodeau S N, Bren G, Schaid D. Microsatellite instability in cancer of the proximal colon. Science 1993; 260: 816-9:
16. Ionov Y, Peinado M A, Malkhosyan S, Shibata D, Perucho M. Ubiquitous somatic mutations in simple repeated sequences revealed a new mechanism for colonic carcinogenesis. Nature 1993; 363: 558-61.
17. Dietmaier W, Wallinger S, Bocker T, Kullmann F, Fishel R, Ruschoff J. Diagnostic microsatellite instability: definition and correlation with mismatch repair protein expression. Cancer Res 1997; 57: 4749-56.
18. Markowitz S, Wang J, Myeroff L, Parsons R, Sun L, Lutterbaugh J, et al. Inactivation of the type II TGF-beta receptor in colon cancer cells with microsatellite instability. Science 1995; 268: 1336-8.
19. Bhattacharyya N P, Ganesh A, Phear G, Richards B, Skandalis A, Meuth M. Molecular analysis of mutations in mutator colorectal carcinoma cell lines. Hum Mol Genet 1995; 4: 2057-64.
20. Salk J J, Salipante S J, Risques R A, Crispin D A, Li L, Bronner M P, et al. Clonal expansions in ulcerative colitis identify patients with neoplasia. Proc Natl Acad Sci USA 2009.
21. Laiho P, Launonen V, Lahermo P, Esteller M, Guo M, Herman J G, et al. Low-level microsatellite instability in most colorectal carcinomas. Cancer Res 2002; 62: 1166-70.
22. Sjoblom T, Jones S, Wood L D, Parsons D W, Lin J, Barber T D, et al. The consensus coding sequences of human breast and colorectal cancers. Science 2006; 314: 268-74.
23. Fox E J, Salk J J, Loeb L A. Cancer genome sequencing—an interim analysis. Cancer Res 2009; 69: 4948-50.
24. Ley T J, Mardis E R, Ding L, Fulton B, McLellan M D, Chen K, et al. DNA sequencing of a cytogenetically normal acute myeloid leukaemia genome. Nature 2008; 456: 66-72.
25. Mardis E R, Ding L, Dooling D J, Larson D E, McLellan M D, Chen K, et al. Recurring mutations found by sequencing an acute myeloid leukemia genome. N Engl J Med 2009; 361: 105 8-66.
26. Pleasance E D, Cheetham R L, Stephens P J, McBride D J, Humphray S J, Greenman C D, et al. A comprehensive catalogue of somatic mutations from a human cancer genome. Nature in press.
27. Pleasance E D, Stephens P J, O'Meara S, McBride D J, Meynert A, Jones D, et al. A small-cell lung cancer genome with complex signature of tobacco exposure. Nature in press.
28. Renan M J. How many mutations are required for tumorigenesis? Implications from human cancer data. Mol Carcinog 1993; 7: 139-46.
29. Fujii H, Marsh C, Cairns P, Sidransky D, Gabrielson E. Genetic divergence in the clonal evolution of breast cancer. Cancer Res 1996; 56: 1493-7.
30. Campbell P J, Pleasance E D, Stephens P J, Dicks E, Rance R, Goodhead I, et al. Subclonal phylogenetic structures in cancer revealed by ultra-deep sequencing. Proc Natl Acad Sci USA 2008; 105: 13081-6.
31. Bielas J H, Loeb L A. Quantification of random genomic mutations. Nat Methods 2005; 2: 285-90.
32. Bielas J H, Loeb K R, Rubin B P, True L D, Loeb L A. Human cancers express a mutator phenotype. Proc Natl Acad Sci USA 2006; 103: 18238-42.
33. Beckman R A, Loeb L A. Genetic instability in cancer: theory and experiment. Semin Cancer Biol 2005; 15: 423-35.
34. O'Brien S, Berman E, Borghaei H, Deangelo D J, Devetten M P, Devine S, et al. NCCN clinical practice guidelines in oncology: chronic myelogenous leukemia. J Natl Compr Canc Netw 2009; 7: 984-1023.
35. Radich J P, Dai H, Mao M, Oehler V, Schelter J, Druker B, et al. Gene expression changes associated with progression and response in chronic myeloid leukemia. Proc Natl Acad Sci USA 2006; 103: 2794-9.
36. Shah N P, Skaggs B J, Branford S, Hughes T P, Nicoll J M, Paquette R L, et al. Sequential ABL kinase inhibitor therapy selects for compound drug-resistant BCR-ABL mutations with altered oncogenic potency. J Clin Invest 2007; 117: 2562-9.
37. Michor F, Hughes T P, Iwasa Y, Branford S, Shah N P, Sawyers C L, et al. Dynamics of chronic myeloid leukaemia. Nature 2005; 435: 1267-70.
38. Hughes T P, Kaeda J, Branford S, Rudzki Z, Hochhaus A, Hensley M L, et al. Frequency of major molecular responses to imatinib or interferon alfa plus cytarabine in newly diagnosed chronic myeloid leukemia. N Engl J Med 2003; 349: 1423-32.
39. Steinkamp M P, O'Mahony O A, Brogley M, Rehman H, Lapensee E W, Dhanasekaran S, et al. Treatment-dependent androgen receptor mutations in prostate cancer exploit multiple mechanisms to evade therapy. Cancer Res 2009; 69: 4434-42.
40. Wogan G N, Hecht S S, Felton J S, Conney A H, Loeb L A. Environmental and chemical carcinogenesis. Semin Cancer Biol 2004; 14: 473-86.
41. Hadziyannis S J. Hepatocellular carcinoma and type B hepatitis. Clin Gastroenterol 1980; 9: 117-34.
42. Nelson W G, De Marzo A M, Deweese T L, Lin X, Brooks J D, Putzi M J, et al. Preneoplastic prostate lesions: an opportunity for prostate cancer prevention. Ann N Y Acad Sci 2001; 952: 135-44.
43. Schottenfeld D, Beebe-Dimmer J. Chronic inflammation: a common and important factor in the pathogenesis of neoplasia. CA Cancer J Clin 2006; 56: 69-83.
44. Ekbom A, Helmick C, Zack M, Adami H O. Ulcerative colitis and colorectal cancer. A population-based study. N Engl J Med 1990; 323: 1228-33.
45. Matsumoto Y, Marusawa H, Kinoshita K, Endo Y, Kou T, Morisawa T, et al. Helicobacter pylori infection triggers aberrant expression of activation-induced cytidine deaminase in gastric epithelium. Nat Med 2007; 13: 470-6.
46. Naugler W E, Karin M. NF-kappaB and cancer-identifying targets and mechanisms. Curr Opin Genet Dev 2008; 18: 19-26.
47. Colotta F, Allavena P, Sica A, Garlanda C, Mantovani A. Cancer-related inflammation, the seventh hallmark of cancer: links to genetic instability. Carcinogenesis 2009; 30: 1073-81.
48. Meira L B, Bugni J M, Green S L, Lee C W, Pang B, Borenshtein D, et al. DNA damage induced by chronic inflammation contributes to colon carcinogenesis in mice. J Clin Invest 2008; 118: 2516-25.
49. Valinluck V, Sowers L C. Inflammation-mediated cytosine damage: a mechanistic link between inflammation and the epigenetic alterations in human cancers. Cancer Res 2007; 67: 5583-6.
50. Coffin J M. HIV population dynamics in vivo: implications for genetic variation, pathogenesis, and therapy. Science 1995; 267: 483-9.
Eigen M. Selforganization of matter and the evolution of biological macromolecules. Naturwissenschaften 1971; 58: 465-523.
52. Eigen M. Error catastrophe and antiviral strategy. Proc Natl Acad Sci USA 2002; 99: 13374-6.
53. Mullins J I, Jensen M A. Evolutionary dynamics of HIV-1 and the control of AIDS. Curr Top Microbiol Immunol 2006; 299: 171-92.
54. Heidelberger C, Chaudhuri N K, Danneberg P, Mooren D, Griesbach L, Duschinsky R, et al. Fluorinated pyrimidines, a new class of tumour-inhibitory compounds. Nature 1957; 179: 663-6.
55. Delaney J C, Essigmann J M. Biological properties of single chemical-DNA adducts: a twenty year perspective. Chem Res Toxicol 2008; 21: 232-52.
56. Janssen A, Kops G J, Medema R H. Elevating the frequency of chromosome mis-segregation as a strategy to kill tumor cells. Proc Natl Acad Sci USA 2009; 106: 19108-13.
57. Lothe R A, Peltomaki P, Meling G I, Aaltonen L A, Nystrom-Lahti M, Pylkkanen L, et al. Genomic instability in colorectal cancer: relationship to clinicopathological variables and family history. Cancer Res 1993; 53: 5849-52.
58. Samowitz W S, Curtin K, Wolff R K, Tripp S R, Caan B J, Slattery M L. Microsatellite instability and survival in rectal cancer. Cancer Causes Control 2009; 20: 1763-8.
59. Corso G, Pedrazzani C, Marrelli D, Pascale V, Pinto E, Roviello F. Correlation of microsatellite instability at multiple loci with long-term survival in advanced gastric carcinoma. Arch Surg 2009; 144: 722-7.
60. Greenman C, Stephens P, Smith R, Dalgliesh G L, Hunter C, Bignell G, et al. Patterns of somatic mutation in human cancer genomes. Nature 2007; 446: 153-8.
61. Parsons D W, Jones S, Zhang X, Lin J C, Leary R J, Angenendt P, et al. An integrated genomic analysis of human glioblastoma multiforme. Science 2008; 321: 1807-12.
62. Comprehensive genomic characterization defines human glioblastoma genes and core pathways. Nature 2008; 455: 1061-8.
63. Ding L, Getz G, Wheeler D A, Mardis E R, McLellan M D, Cibulskis K, et al. Somatic mutations affect key pathways in lung adenocarcinoma. Nature 2008; 455: 1069-75.
64. Pitsikas P, Patapas J M, Cupples C G. Mechanism of 2-aminopurine-stimulated mutagenesis in Escherichia coli. Mutat Res. 2004 Jun. 4; 550(1-2):25-32.
65. Aida M, Yamane K, Nagata C. An explanation of the induction of mutations by 2-aminopurine from an ab initio molecular orbital study. Mutat Res. 1986 January; 173(1):49-54.
66. Inoue, H.; Imura, A.; Ohtsuka, E. Nucleic Acids Res. 1985, 13, 7119-7128
67. Staubli, A. B.; Dervan, P. B. Nucleic Acids Res. 1994, 22, 2637-2642.
68. Durland, R. H.; Rao, T. S.; Jayaraman, K.; Revankar, G. R. Bioconjugate Chem. 1995, 6, 278-282.
69. Nordmann P L, Makris J C, Reznikoff W S. Inosine induced mutations. Mol Gen Genet. 1988 September; 214(1):62-7. PubMed PMID: 2852303.
70. Meuth, M. The molecular basis of mutations induced by deoxyribonucleoside triphosphate pool imbalances in mammalian cells. Exp. Cell Res. 1989 April; 181(2), 305
71. Pleasance E D, Stephens P J, O'Meara S, McBride D J, Meynert A, Jones D, et al. A small-cell lung cancer genome with complex signatures of tobacco exposure. Nature 2010; 463:184-90.
72. Pleasance E D, Cheetham R K, Stephens P J, McBride D J, Humphray S J, Greenman C D, et al. A comprehensive catalogue of somatic mutations from a human cancer genome. Nature 2010; 463:191-6.
73. Ding L, Ellis M J, Li S, Larson D E, Chen K, Wallis J W, et al. Genome remodelling in a basal-like breast cancer metastasis and xenograft. Nature 2010; 464:999-1005.
74. Lee W, Jiang Z, Liu J, Haverty P M, Guan Y, Stinson J, et al. The mutation spectrum revealed by paired genome sequences from a lung cancer patient. Nature 2010; 465:473-7.
75. Roach J C, Glusman G, Smit A F, Huff C D, Hubley R, Shannon P T, et al. Analysis of genetic inheritance in a family quartet by whole-genome sequencing. Science 2010; 328:636-9.

### REFERENCE II

Boveri T. Uber mehrpolige mitosen als mittel zur analyse des zellkerns. Verh D Pliys Med Ges Wurzberg 1902; 35:67-90.
von Hansemann D. Ueber asymmetrische Zelltheilung in epithel Krebsenund deren biologische Bedeutung. Virchows Arch A Pathol Anat Histopathol 1890; 119:299.
Pollack J R, Perou C M, Alizadeh A A, Eisen M B, Pergamenschikov A, Williams C F, et al. Genome-wide analysis of DNA copy-number changes using cDNA microarrays. Nat Genet 1999; 23:41-6.
Schrock E, du Manoir S, Veldman T, Schoell B, Wienberg J, Ferguson-Smith M A, et al. Multicolor spectral karyotyping of human chromosomes. Science 1996; 273:494-7.
Klein C A, Schmidt-Kittler O, Schardt J A, Pantel K, Speicher M R, Riethmuller G. Comparative genomic hybridization, loss of heterozygosity, and DNA sequence analysis of single cells. Proc Natl Acad Sci USA 1999; 96:4494-9.
Stoler D L, Chen N, Basik M, Kahlenberg M S, Rodriguez-Bigas M A, Petrelli N J, et al. The onset and extent of genomic instability in sporadic colorectal tumor progression. Proc Natl Acad Sci USA 1999; 96:15121-6.
Fishel R, Lescoe M K, Rao M R, Copeland N G, Jenkins N A, Garber J, et al. The human mutator gene homolog MSH2 and its association with hereditary nonpolyposis colon cancer. Cell 1993; 75:1027-38.
Parsons R, Li G M, Longley M J, Fang W H, Papadopoulos N, Jen J, et al. Hypermutability and mismatch repair deficiency in RER+ tumor cells. Cell 1993; 75:1227-36.
Dietmaier W, Wallinger S, Bocker T, Kullmann F, Fishel R, Ruschoff J. Diagnostic microsatellite instability: definition and correlation with mismatch repair protein expression. Cancer Res 1997; 57:4749-56.
Brentnall T A, Crispin D A, Bronner M P, Cheman S P, Hueffed M, Rabinovitch P S, et al. Microsatellite instability in nonneoplastic mucosa from patients with chronic ulcerative colitis. Cancer Res 1996; 56:1237-40.
Kondo A, Safaei R, Mishima M, Niedner H, Lin X, Howell S B. Hypoxia-induced enrichment and mutagenesis of cells that have lost DNA mismatch repair. Cancer Res 2001; 61:7603-7.
Salk J J, Salipante S J, Risques R A, Crispin D A, Li L, Bronner M P, et al. Clonal expansions in ulcerative colitis identify patients with neoplasia. Proc Natl Acad Sci USA 2009.
Cleaver J E. Defective repair replication of DNA in xeroderma pigmentosum. Nature 1968; 218:652-6.
Al-Tassan N, Chmiel N H, Maynard J, Fleming N, Livingston A L, Williams G T, et al. Inherited variants of MYH associated with somatic G:C→T:A mutations in colorectal tumors. Nat Genet 2002; 30:227-32.
King M C, Marks J H, Mandell J B. Breast and ovarian cancer risks due to inherited mutations in BRCA1 and BRCA2. Science 2003; 302:643-6.
Riballo E, Critchlow S E, Teo S H, Doherty A J, Priestley A, Broughton B, et al. Identification of a defect in DNA ligase IV in a radiosensitive leukaemia patient. Curr Biol 1999; 9:699-702.
Masutani C, Araki M, Yamada A, Kusumoto R, Nogimori T, Maekawa T, et al. Xeroderma pigmentosum variant (XP-V) correcting protein from HeLa cells has a thymine dimer bypass DNA polymerase activity. EMBO J 1999; 18:3491-501.
Malkin D, Li F P, Strong L C, Fraumeni Jr J F, Nelson C E, Kim D H, et al. Germ line p53 mutations in a familial syndrome of breast cancer, sarcomas, and other neoplasms. Science 1990; 250:1233-8.
Srivastava S, Zou Z Q, Pirollo K, Blattner W, Chang E H. Germ-line transmission of a mutated p53 gene in a cancer-prone family with Li-Fraumeni syndrome. Nature 1990; 348:747-9.
Goode E L, Ulrich C M, Potter J D. Polymorphisms in DNA repair genes and associations with cancer risk. Cancer Epidemiol Biomarkers Prev 2002; 11:1513-30.
Loeb L A, Bielas J H, Beckman R A. Cancers exhibit a mutator phenotype: clinical implications. Cancer Res 2008; 68:3551-7 [discussion 7].
Bielas J H, Loeb K R, Rubin B P, True L D, Loeb L A. Human cancers express a mutator phenotype. Proc Natl Acad Sci USA 2006; 103:18238-42.
Zheng L, Dai H, Zhou M, Li M, Singh P, Qiu J, et al. Fen1 mutations result in autoimmunity, chronic inflammation and cancers. Nat Med 2007; 13:812-9.
Comprehensive genomic characterization defines human glioblastoma genes and core pathways. Nature 2008; 455:1061-8.
Ding L, Ellis M J, Li S, Larson D E, Chen K, Wallis J W, et al. Genome remodelling in a basal-like breast cancer metastasis and xenograft. Nature 2010; 464:999-1005.
Ding L, Getz G, Wheeler D A, Mardis E R, McLellan M D, Cibulskis K, et al. Somatic mutations affect key pathways in lung adenocarcinoma. Nature 2008; 455:1069-75.
Greenman C, Stephens P, Smith R, Dalgliesh G L, Hunter C, Bignell G, et al. Patterns of somatic mutation in human cancer genomes. Nature 2007; 446:153-8.
Jones S, Zhang X, Parsons D W, Lin J C, Leary R J, Angenendt P, et al. Core signaling pathways in human pancreatic cancers revealed by global genomic analyses. Science 2008; 321:1801-6.
Lee W, Jiang Z, Liu J, Haverty P M, Guan Y, Stinson J, et al. The mutation spectrum revealed by paired genome sequences from a lung cancer patient. Nature 2010; 465:473-7.
Ley T J, Mardis E R, Ding L, Fulton B, McLellan M D, Chen K, et al. DNA sequencing of a cytogenetically normal acute myeloid leukaemia genome. Nature 2008; 456:66-72.
Mardis E R, Ding L, Dooling D J, Larson D E, McLellan M D, Chen K, et al. Recurring mutations found by sequencing an acute myeloid leukemia genome. N Engl J Med 2009; 361:1058-66.
Pleasance E D, Cheetham R K, Stephens P J, McBride D J, Humphray S J, Greenman C D, et al. A comprehensive catalogue of somatic mutations from a human cancer genome. Nature 2010; 463:191-6.
Pleasance E D, Stephens P J, O'Meara S, McBride D J, Meynert A, Jones D, et al. A small-cell lung cancer genome with complex signatures of tobacco exposure. Nature 2010; 463:184-90.
Sjoblom T, Jones S, Wood L D, Parsons D W, Lin J, Barber T D, et al. The consensus coding sequences of human breast and colorectal cancers. Science 2006; 314:268-74.
Wood L D, Parsons D W, Jones S, Lin J, Sjoblom T, Leary R J, et al. The genomic landscapes of human breast and colorectal cancers. Science 2007; 318:1108-13.
Hudson T J, Anderson W, Artez A, Barker A D, Bell C, Bernabe R R, et al. International network of cancer genome projects. Nature 2010; 464:993-8.
Rubin A F, Green P. Mutation patterns in cancer genomes. Proc Natl Acad Sci USA 2009; 106:21766-70.
McMurray C T. DNA secondary structure: a common and causative factor for expansion in human disease. Proc Natl Acad Sci USA 1999; 96:1823-5.
Fox E J, Salk J J, Loeb L A. Cancer genome sequencing—an interim analysis. Cancer Res 2009; 69:4948-50.
Balducci L, Khansur T, Smith T, Hardy C. Prostate cancer: a model of cancer in the elderly. Arch Gerontol Geriatr 1989; 8:165-87.
Beasley R P, Hwang L Y, Lin C C, Chien C S. Hepatocellular carcinoma and hepatitis B virus. A prospective study of 22 707 men in Taiwan. Lancet 1981; 2:1129-33.
Kirk G D, Bah E, Montesano R. Molecular epidemiology of human liver cancer: insights into etiology, pathogenesis and prevention from The Gambia, West Africa. Carcinogenesis 2006; 27:2070-82.
Schuster P. RNA based evolutionary optimization. Orig Life Evol Biosph 1993; 23:373-91.
Cahill D P, Kinzler K W, Vogelstein B, Lengauer C. Genetic instability and Darwinian selection in tumours. Trends Cell Biol 1999; 9:M57-60.
Loeb L A, Loeb K R, Anderson J P. Multiple mutations and cancer. Proc Natl Acad Sci USA 2003; 100:776-81.
Sole R V, Deisboeck T S. An error catastrophe in cancer? J Theor Biol 2004; 228:47-54.
Bull J J, Wilke C O. Lethal mutagenesis of bacteria. Genetics 2008; 180:1061-70.
Glaab W E, Mitchell L S, Miller J E, Vlasakova K, Skopek T R. 5-Fluorouracil forward mutation assay in Salmonella: determination of mutational target and spontaneous mutational spectra. Mutat Res 2005; 578:238-46.
Skopek T R, Thilly W G. Rate of induced forward mutation at 3 genetic loci in Salmonella typhimurium. Mutat Res 1983; 108:45-56.
Rechkoblit O, Kolbanovskiy A, Malinina L, Geacintov N E, Broyde S, Patel D J. Mechanism of error-free and semitargeted mutagenic bypass of an aromatic amine lesion by Y-family polymerase Dpo4. Nat Struct Mol Biol 2010; 17:379-88.
Upton D C, Wang X, Blans P, Perrino F W, Fishbein J C, Akman S A. Mutagenesis by exocyclic alkylamino purine adducts in Escherichia coli. Mutat Res 2006; 599:1-10.
Amer E S, Eriksson S. Mammalian deoxyribonucleoside kinases. Pharmacol Ther 1995; 67:155 86.
Reichard P. Interactions between deoxyribonucleotide and DNA synthesis. Annu Rev Biochem 1988; 57:349-74.
Mo J Y, Maki H, Sekiguchi M. Hydrolytic elimination of a mutagenic nucleotide, 8-oxodGTP, by human 18-kilodalton protein: sanitization of nucleotide pool. Proc Natl Acad Sci USA 1992; 89:11021-5.
Lemee F, Bergoglio V, Fernandez-Vidal A, Machado-Silva A, Pillaire M J, Bieth A, et al. DNA polymerase theta up-regulation is associated with poor survival in breast cancer, perturbs DNA replication, and promotes genetic instability. Proc Natl Acad Sci USA 2010; 107:13390-5.
Darby G, Larder B A. The clinical significance of antiviral drug resistance. Res Virol 1992; 143:116-20.
Hochhaus A, Hughes T. Clinical resistance to'imatinib: mechanisms and implications. Hematol Oncol Clin North Am 2004; 18:641-56, ix.
Larder B A, Darby G, Richman D D. HIV with reduced sensitivity to zidovudine (AZT) isolated during prolonged therapy. Science 1989; 243:1731-4.
Shah N P, Tran C, Lee F Y, Chen P, Norris D, Sawyers C L. Overriding imatinib resistance with a novel ABL kinase inhibitor. Science 2004; 305:399-401.
Weisberg E, Manley P W, Breitenstein W, Bruggen J, Cowan-Jacob S W, Ray A, et al. Characterization of AMN107, a selective inhibitor of native and mutant Bcr-Abl. Cancer Cell 2005; 7:129-41.
Agudo R, Ferrer-Orta C, Arias A, de la Higuera I, Perales C, Pérez-Luque R, et al. A multi-step process of viral adaptation to a mutagenic nucleoside analogueby modulation of transition types leads to extinction-escape. PLoS Pathog 2010; 6:e1001072.
Baker D B, Landrigan P J. Occupationally related disorders. Med Clin North Am 1990; 74:441-60.
Wogan G N, Hecht S S, Felton J S, Connney A H, Loeb L A. Environmental and chemical carcinogenesis. Semin Cancer Biol 2004; 14:473-86.
Salk J J, Horwitz M S. Passenger Mutations as a Marker of Clonal Cell Lineages in Emerging Neoplasia. Seminars in Cancer Biology 2010; 20:294-303.
Beckman R A. Efficiency of carcinogenesis: Is the mutator phenotype inevitable? Seminars in Cancer Biology 2010; 20:340-52.
Hoffmann J S, Cazaux C. Aberrant expression of alternative DNA polymerases: A source of mutator phenotype as well as replicative stress in cancer. Seminars in Cancer Biology 2010; 20:312-9.
Anderson J P, Daifuku R, Loeb L A. Viral error catastrophe by mutagenic nucleosides. Annu Rev Microbiol 2004; 58:183-205.
Prindle M J, Fox E J, Loeb L A. The mutator phenotype in cancer: molecular mechanisms and targeting strategies. Curr Drug Targets. 2010 October; 11(10):1296-303.
Fox E J, Loeb L A. Lethal mutagenesis: targeting the mutator phenotype in cancer. Semin Cancer Biol. 2010 October; 20(5):353-9.

It is understood that the foregoing detailed description and examples are illustrative only and are not to be taken as limitations upon the scope of the invention. Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method of increasing mutation frequency in a cancer cell genome, comprising contacting a cancer cell with one or more mutagenic compounds, wherein said one or more mutagenic compounds increase the mutation frequency of a cancer cell or progeny cell thereof by at least two-fold relative to the mutation frequency of the cancer cell or progeny cell thereof when not contacted by the one or more mutagenic compounds.

2. The method of claim 1, wherein the increase in mutation frequency of the cancer cell or progeny cell thereof upon said contacting causes lethal mutagenesis of the cancer cell or progeny cell thereof.

3. The method of claim 1, wherein said one or more mutagenic compounds do not inhibit replication or extension of a nucleic acid by a DNA polymerase.

4. The method of claim 1, wherein at least one of the one or more mutagenic compounds is incorporated into the genome of the cancer cell or progeny cell thereof by a DNA polymerase in the cancer cell.

5. The method of claim 1, wherein at least one of the one or more mutagenic compounds increases the mutation frequency of the cancer cell by increasing non-complementary nucleoside mispairings in the cancer cell.

6. The method of claim 1, wherein the one or more mutagenic compounds comprise a mutagenic nucleoside analog.

7. A method of treating cancer in a subject in need thereof, the method comprising administering to a subject having a cancer or a tumor a pharmaceutical composition comprising at least one mutagenic compound, wherein said at least one mutagenic compound increases the mutation frequency of a cancer cell or progeny cell thereof in said subject by at least two-fold relative to the mutation frequency of the cancer cell or progeny cell in the subject when not administered the pharmaceutical composition comprising at least one mutagenic compound.

8. The method of claim 7, wherein the increase in mutation frequency of the cancer cell or progeny cell thereof upon said administering causes lethal mutagenesis of the cancer cell or progeny cell thereof.

9. The method of claim 7, wherein the at least one mutagenic compound does not inhibit replication or extension of a nucleic acid by a mammalian DNA polymerase.

10. The method of claim 7, wherein the at least one mutagenic compound is incorporated into the genome of the cancer cell or progeny cell thereof in said subject by a human cellular DNA polymerase.

11. The method of claim 7, wherein the at least one mutagenic compound increases the mutation frequency of the cancer cell or progeny cell thereof in the subject by increasing non-complementary nucleoside mispairings in the cancer cell.

12. The method of claim 7, wherein the at least one mutagenic compound comprises a mutagenic nucleoside analog.

13. The method of claim 7, further comprising administering to the subject a different anti-cancer agent.

14. The method of claim 7, wherein said cancer has been diagnosed as therapy-resistant or as non-responsive to all therapies tested.

15. The method of claim 7, wherein said cancer has been diagnosed as only responsive to highly toxic chemotherapeutic agents.

16. The method of claim 7, wherein said cancer is highly likely to recur in less than one year.

17. The method of claim 7, wherein said cancer has metastasized to multiple sites some or all of which are not responsive to therapy.

18. The method of claim 7, wherein said type of cancer is known to be highly non-responsive to all types of anti-cancer agents.

19. The method of claim 7, wherein said cancer has repeatedly recurred after treatments with multiple anti-cancer agents.
